(19) United States
(12) Patent Application Publication
(10) Pub. No.: US 2025/0032755 A1
(43) Pub. Date: Jan. 30, 2025

(54) TUBE SECUREMENT ASSEMBLY AND USES THEREOF

(71) Applicant: Children's National Medical Center, Washington, DC (US)

(72) Inventors: Anthony SANDLER, Washington, DC (US); Reza MONFAREDI, Washington, DC (US); Michele SARUWATARI, Washington, DC (US)

(73) Assignee: Children's National Medical Center, Washington, DC (US)

(21) Appl. No.: 18/714,675

(22) PCT Filed: Dec. 9, 2022
(86) PCT No.: PCT/US2022/052394

§ 371 Date: May 30, 2024

Related U.S. Application Data
(60) Provisional application No. 63/265,170, filed on Dec. 9, 2021.

(51) Int. Cl. A61M 25/02

(52) U.S. Cl. CPC A61M 25/02; A61M 2025/0213

(57) ABSTRACT

A securement assembly for a medical catheter inserted into a human body, including an assembly body and a domed cap removably coupled to the assembly body, the domed cap having a first half and an opposing second half, the first half and the second half being coupled together to form a channel inside the domed cap terminating at an opening in the domed cap, the channel being configured to secure the medical catheter, and the assembly body securing an area of the human body surrounding the insertion site of the medical catheter.

### CROSS-REFERENCE TO RELATED APPLICATIONS

The present application claims priority to U.S. Provisional Application No. 63/265,170, filed Dec. 9, 2021, which is incorporated herein by reference in its entirety for all purposes.

### BACKGROUND

### FIELD OF THE DISCLOSURE

The present disclosure pertains to securement devices for securing a tube, such as a medical catheter which is inserted into the body for passage of fluids into or out of the body.

### DESCRIPTION OF THE RELATED ART

Medical catheters are inserted into the body to drain fluids from the body or deliver fluids into the body. There is a need to properly secure a medical catheter to prevent movement relative to its insertion site and to maintain a desired depth of insertion.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present invention.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a securement assembly for a medical catheter inserted into an organ, comprising a cylindrical body; and a domed cap removably coupled to the cylindrical body, the domed cap having a first half and an opposing second half, the first half and the opposing second half being removably coupled together to form a channel inside the domed cap terminating at an opening in the domed cap, the channel being configured to secure the medical catheter, and the cylindrical body forming a chamber inside of the cylindrical body, the chamber being configured to secure the organ.

In one embodiment, the present disclosure is related to a securement assembly for a medical catheter inserted into an organ, comprising a cylindrical body; and a domed cap removably coupled to the cylindrical body, the domed cap having a first half and an opposing second half, the first half and the opposing second half being removably coupled together to form a channel inside the domed cap terminating at an opening in the domed cap, the channel being configured to secure the medical catheter, the cylindrical body forming a chamber inside of the cylindrical body, the chamber being configured to secure the organ, and the domed cap being a first material and the cylindrical body being a second material, wherein the second material is more flexible than the first material.

In one embodiment, the present disclosure is related to a securement assembly for a medical catheter inserted into an organ, comprising a cylindrical body; and a domed cap removably coupled to the cylindrical body, the domed cap having a first half and an opposing second half, the first half and the opposing second half being removably coupled together to form a channel inside the domed cap terminating at an opening in the domed cap, the channel being configured to secure the medical catheter, the cylindrical body having a layer of padding forming an inner wall of the cylindrical body, and the cylindrical body forming a chamber inside of the cylindrical body, the chamber being configured to secure the organ.

In one embodiment, the present disclosure is related to a securement assembly for a medical catheter inserted into an opening in skin, comprising a dock being attached to a base; and a domed cap removably coupled to the dock, the dock surrounding a cutout in the base, the domed cap having a first half and an opposing second half, the first half and the opposing second half being removably coupled together to form a channel inside the domed cap terminating at an opening in the domed cap, the channel being configured to secure the medical catheter, and the domed cap including a docking structure, wherein the docking structure fits into a slot in the dock to couple the domed cap to the dock.

In one embodiment, the present disclosure is related to a securement assembly for a medical catheter inserted into an opening in skin, comprising a dock being attached to a base; and a domed cap removably coupled to the dock, the dock surrounding a cutout in the base, the domed cap having a first half and an opposing second half, the first half and the opposing second half being coupled together to form a channel inside the domed cap terminating at an opening in the domed cap, the channel being configured to secure the medical catheter, the opening in the domed cap being perpendicular to the base, the domed cap including a docking structure, wherein the docking structure fits into a slot in the dock to couple the domed cap to the dock.

In one embodiment, the present disclosure is related to a securement assembly for a medical catheter inserted into an opening in skin, comprising a dock being attached to a base; and a domed cap removably coupled to the dock, the dock surrounding a cutout in the base, and the domed cap having a first half and an opposing second half, the first half and the opposing second half being removably coupled together to form a channel inside the domed cap terminating at an opening in the domed cap, the channel being configured to secure the medical catheter, the domed cap being a first material and the base being a second material, wherein the second material is more flexible than the first material.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a securement assembly for a medical catheter, according to one embodiment of the present disclosure;

FIG. 2 is a securement assembly for a medical catheter, according to one embodiment of the present disclosure;

FIG. 3A is a securement assembly for a medical catheter, according to one embodiment of the present disclosure;

FIG. 3B is a top view of a securement assembly for a medical catheter, according to one embodiment of the present disclosure;

FIG. 4A is a cross-section of a cap of a securement assembly for a medical catheter, according to one embodiment of the present disclosure;

FIG. 4B is a cap of a securement assembly for a medical catheter, according to one embodiment of the present disclosure;

FIG. 5 is a padding layer for a securement assembly for a medical catheter, according to one embodiment of the present disclosure;

FIG. 6 is an anchoring track for a securement assembly for a medical catheter, according to one embodiment of the present disclosure;

FIG. 7 is a side view of a securement assembly for a medical catheter, according to one embodiment of the present disclosure;

FIG. 8 is a method for attaching a securement assembly for a medical catheter, according to one embodiment of the present disclosure;

FIG. 9 is a securement assembly for a medical catheter, according to one embodiment of the present disclosure;

FIG. 10 is a body of a securement assembly for a medical catheter, according to one embodiment of the present disclosure;

FIG. 11A is a cap of a securement assembly for a medical catheter in an open position, according to one embodiment of the present disclosure;

FIG. 11B is a cap of a securement assembly for a medical catheter in a closed position, according to one embodiment of the present disclosure;

FIG. 11C is a cap of a securement assembly for a medical catheter in a closed position, according to one embodiment of the present disclosure;

FIG. 12 is a securement assembly for a medical catheter, according to one embodiment of the present disclosure;

FIG. 13 is a body of a securement assembly for a medical catheter, according to one embodiment of the present disclosure;

FIG. 14 is a cap of a securement assembly for a medical catheter, according to one embodiment of the present disclosure;

FIG. 15 is a method for attaching a securement assembly for a medical catheter, according to one embodiment of the present disclosure; and

FIG. 16 is a method for removing the securement assembly for a medical catheter, according to one embodiment of the present disclosure.

### DETAILED DESCRIPTION OF THE INVENTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one embodiment, the present disclosure is directed towards a securement assembly for a medical catheter, the medical catheter being, for example, a urinary catheter, an abdominal catheter, a thoracic catheter, an endotracheal tube, a feeding tube, a nasogastric tube, an orogastric tube, a laryngeal mask airway tube, a tracheostomy tube, a Jackson-Pratt drain, a Davol drain, a gastrostomy tube, a peritoneal tube, a T-tube, a rectal tube, an angiocatheter, a central line, a tunneled catheter, buttons, or the like. A medical catheter can be inserted into the body as a non-surgical intervention to drain fluid from or administer fluid to the body. In some conditions, a medical catheter can be used to treat infection, bleeding, or leakage of pulmonary or gastrointestinal air or fluid within the body. Medical catheters can also be used as part of a surgical procedure or to prevent postoperative complications. A urinary catheter is an example of a medical catheter that can be inserted into a patient's urethra to drain the bladder before or after a radical prostatectomy, which is performed to treat prostate cancer. The urinary catheter is typically left in place or regularly replaced over a period of one to two weeks to allow the bladder and urethra to properly heal after the procedure. The positioning of a medical catheter and various characteristics of the medical catheter (e.g., caliber, size, flexibility, drainage type, means of suction) are variable and dependent on the type and usage of the medical catheter. Medical catheters can be inserted into the body temporarily, for a number of days or weeks, or even indefinitely.

It is important that a medical catheter is properly secured at or near the insertion site in order to prevent complications, including, but not limited to, improper drainage, malpositioning, tissue damage, fistulation, infection, pain, or trauma to the skin or tissue. The medical catheter should be secured within the insertion site such that it does not move independently of the body or relative to the site or organ of insertion. The insertion site can be an orifice of the body or can be an incision or opening made in the skin. Maintaining a known depth and location of an internal drain opening at the end of the medical catheter that is inserted into the body is crucial to ensure proper drainage and to avoid serious complications. It is also important to secure the medical catheter at the insertion site (also referred to as the exit site) to prevent pain or trauma, including infection. The tubing can rub against the skin and cause irritation when the medical catheter moves in and out of the insertion site or when the angle of insertion changes. Proper securement of the medical catheter can include securement while the patient is in motion. In certain cases, patients expect to have full mobility and to be able to maintain their typical lifestyles while the medical catheter is inserted. Therefore, the securement of the medical catheter should prevent any independent movement of the medical catheter throughout a wide range of patient motion and activity. In many use cases, there may be a wound at or near the insertion site. The wound may need to be accessible in order to monitor healing, apply medication, change a dressing, etc. while still protecting the wound from exposure or irritation that may lead to infection. In addition, a catheter may need to be regularly removed or replaced in order to remove drainage, maintain patient hygiene, and provide access to insertion site or surrounding areas.

A conventional method for securing a medical catheter is to suture the tubing to the skin at the exit site of the medical catheter, often with multiple throws through the patient's skin. These sutures can be uncomfortable and can eventually pull through the skin, causing tissue damage and releasing the tubing from the suture site. Adhesive dressings and medical-grade tapes are also suboptimal as they are typically applied in a piecemeal fashion and do not completely secure the medical catheter in place, resulting in increased risk of drain dislodgement and patient discomfort. Adhesives are also cumbersome to remove and reapply on a regular basis and may lead to irritation or reduced efficacy over time.

In some embodiments, the present disclosure is directed towards a medical catheter securement assembly including a first component for securing the medical catheter and a second component for attachment to or securing of the skin, organ, or body part at or near the insertion site. The first component can be referred to herein as a cap, a first holding portion, a first holding component, or a medical catheter holder. The second component can be referred to herein as a body, a second holding portion, a second holding component, or an organ attachment. The first component and the second component can be removably coupled to each other in order to secure a medical catheter at the insertion site. The securement assembly can prevent movement of the medical catheter relative to the insertion site and the surrounding body parts. In some embodiments, the securement assembly can absorb shock from impact or force on the body to prevent movement of the medical catheter. In some embodiments, each of the first component and the second component can include anchors, fasteners, aligners, locking mechanisms, and/or attachments, which will be described in greater detail herein. The first component can secure medical catheters of varying diameters, thicknesses, lengths, materials, orientations, and angles in a channel or opening. The second component can be securely fitted or fixed to a human body part, appendage, or organ where the medical catheter has been inserted. The human organ can include an area of skin. The coupling of the first component and the second component can secure the medical catheter while reducing unwanted contact between the securement assembly and a wound or insertion site. In one embodiment, the second component can remain fixed to the human organ while the first component is detached from the second component to provide access to the insertion site and the medical catheter. The separation of the first component and the second component can enable the medical catheter to be exchanged and the insertion site or surrounding area to be treated. The first component can then be easily reattached to the second component to continue securing the medical catheter.

In one embodiment, the securement assembly of the present disclosure can be used to secure a urinary catheter. The urinary catheter can be inserted into the penis and through the urethra so that urine can be drained directly from the bladder through the catheter. Urinary catheters can be intermittent (inserted until the bladder is emptied) or indwelling (inserted and held in place for days or weeks). A Foley catheter is an example of an indwelling urinary catheter that is secured inside the bladder with an inflatable balloon. Urinary catheters carry significant risk of urinary tract infection, which can lead to blood infections (sepsis), as well as urethral injury or skin breakdown. Various external and internal forces, including patient movement and the weight of an attached drainage bag, can cause tugging or movement of the urinary catheter, resulting in trauma to the urinary tract and/or external genitalia. Pain and discomfort while a urinary catheter is inserted can affect medical care and patient outcomes. Properly securing a urinary catheter at the insertion site, or the tip of the penis, can prevent movement of the urinary catheter independent of movement of the penis and can improve usage of the urinary catheter.

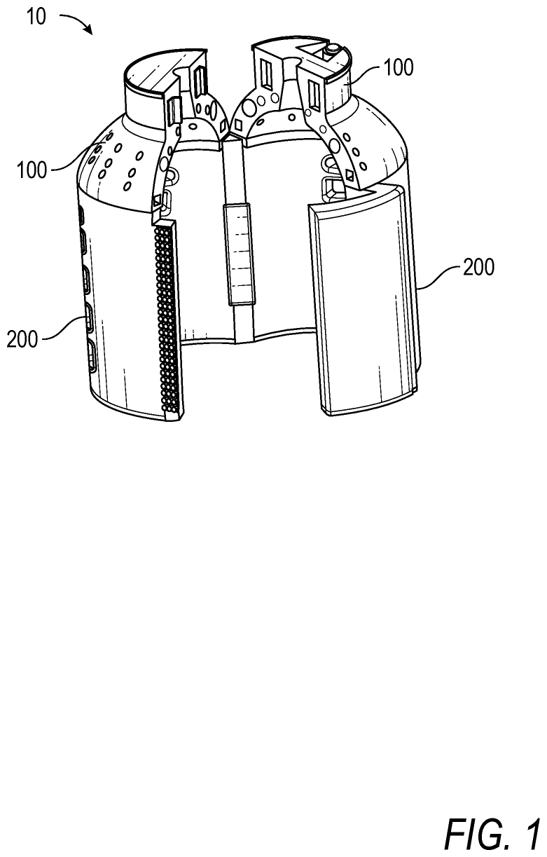

FIG. 1 is an illustration of a securement assembly 10 for a medical catheter, according to one embodiment of the present disclosure. In some embodiments, the securement assembly of FIG. 1 can be used to secure a urinary catheter inserted into a penis. The securement assembly of FIG. 1 can include a domed cap 100, wherein the cap 100 can secure a urinary catheter inserted into the penis, and a cylindrical body 200, wherein the body 200 can be attached to or around the penis. The cap 100 and the body 200 can be removably coupled together to secure the urinary catheter at the insertion site. In one embodiment, the cap 100 can include one or more components or parts. For example, the cap 100 can include two halves, wherein the halves are attached at an anchor point. The two halves can be substantially similar in design and size. In one example, the anchor point can be a hinge or a joint. In one embodiment, each half can pivot or swivel around an axis of rotation formed by the anchor point to open and close the cap. Each half of the cap 100 can be attached to the body 200. The first half of the cap and the second half of the cap can fit together and/or can be fastened together to secure a medical catheter in the cap 100. In one embodiment, the two halves of the cap 100 can include alignment features, locking features, and/or fastening mechanisms to secure the cap in a closed position.

In one embodiment, the body 200 can include one or more components, or parts. For example, the body 200 can include two halves, wherein the halves are attached at an anchor point. The two halves can be substantially similar in design and size. In one example, the anchor point can be a hinge or joint. In one embodiment, the anchor point can be a pin. Each half can pivot or swivel around an axis of rotation formed by the anchor point to open and close the body.

The anchor points of the cap 100 and the anchor points of the body 200 can be separate anchor points or can be the same anchoring mechanism. The two halves of the body 200 can fit together and/or can be fastened together to secure a human organ inside the body 200. For example, the body 200 can fit around a penis with an inserted urinary catheter. The body 200 can fit around the full organ or a portion of the organ, such as the top half or top third. In one embodiment, the two halves of the body 200 can include alignment features, locking features, and/or fastening mechanisms to secure the body in a closed position with the organ inside. In one embodiment, the body 200 can be formed as a single component. The body 200 can be, for example, a flexible component that can be fitted over and around a human organ.

In one embodiment, the securement assembly 10 can be composed of plastic. In one embodiment, the securement assembly 10 can be composed of a biocompatible material or can include at least one biocompatible layer. For example, the inner surfaces of the securement assembly 10 or any surface in contact with the human organ can be coated with a biocompatible silicon layer. In one embodiment, the cap 100 can be composed of a more rigid material than the body 200 to limit movement of the medical catheter secured inside the cap 100. The body 200 of the securement assembly 10 can be more flexible than the cap 100 to enable a range of movement of the human organ. For example, the cap 100 can be a rigid plastic, while the body 200 is made of a flexible material such as silicon. In an exemplary embodiment, the cap 100 can be polylactic acid (PLA) or acrylonitrile butadiene styrene (ABS), which are plastics that can be used for 3D printing. In one embodiment, the cap 100 can be polyethylene. In an exemplary implementation, the cap 100 can be formed by injection molding using any suitable material. In one embodiment, the inner surface of the cap 100 can include a biocompatible and/or flexible material layer. The body 200 can be a silicon or silicone rubber. In one embodiment, the securement assembly 10 can include one or more layers of padding or sponge material for increased comfort and flexibility of certain components.

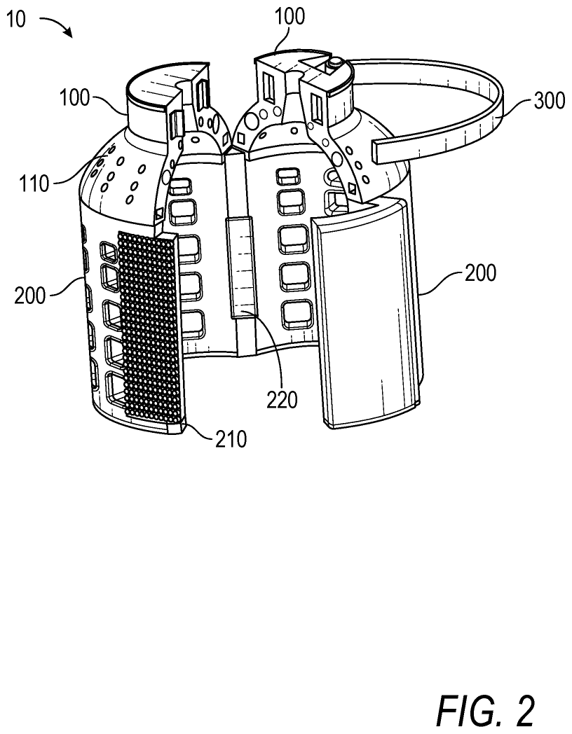

FIG. 2 is an illustration of a securement assembly 10 for a medical catheter in an open position, according to one embodiment of the present disclosure. In one embodiment, the cap 100 can include a base 110. In one embodiment, the base 110 can be a rigid base. The base 110 can be shaped to fit around the end or tip of an organ, such as the penis. In one embodiment, the inner surface of the base 110 can be lined with a biocompatible material, such as a biocompatible silicon. In one embodiment, the cap 100 of the securement assembly 10 can include a fastener 300. In one embodiment, the fastener 300 can be a strip of fabric, flexible plastic, or similar material, wherein the strip can wrap around the top of the cap 100 to secure the first half of the cap and the second half of the cap together. In some embodiments, the fastener 300 can be an elastic strip. The fastener 300 can include a fastening mechanism such as hook-and-loop fabric, an adhesive, a button, a snap fastener, a pin or rivet, or the like in order to secure the cap 100 in a closed position wherein the first half and the second half of the cap are aligned and in contact. The fastener 300 can ensure that the medical catheter is secured tightly in the opening in the center of the cap 100 when the cap is in the closed position. The opening can be an opening of a cylindrical channel running through the cap, wherein the medical catheter can be held in place in the cylindrical channel when the cap is in a closed position. The tight fit of the cap around the medical catheter can reduce unwanted movement or bending of the medical catheter.

In one embodiment, the body 200 of the securement assembly 10 can include a fastening mechanism 210, wherein the fastening mechanism 210 can secure the two halves of the body 200 in a closed position wherein the first half and the inner wall of the second half are aligned and in contact with each other. The two halves of the body 200 can be closed around a human body part, such as the penis. Non-limiting examples of the fastening mechanism 210 can include a strip of hook-and-loop fabric, an adhesive, a button, snap fasteners, pins or rivets, or the like. In one embodiment, the fastening mechanism 210 can be a pressure-activated fastener. In one embodiment, the body 200 of the securement assembly can include an anchor point. An exemplary anchor point 220 is pictured in FIG. 2. In one embodiment, the anchor point 220 can hold the first half of the body 200 and the second half of the body 200 together. Each half of the body can pivot or swivel around an axis of rotation formed by the anchor point 220 to open and close the body 200. In one embodiment, a first half of the cap can be attached to a first half of the body, and a second half of the cap can be attached to a second half of the body.

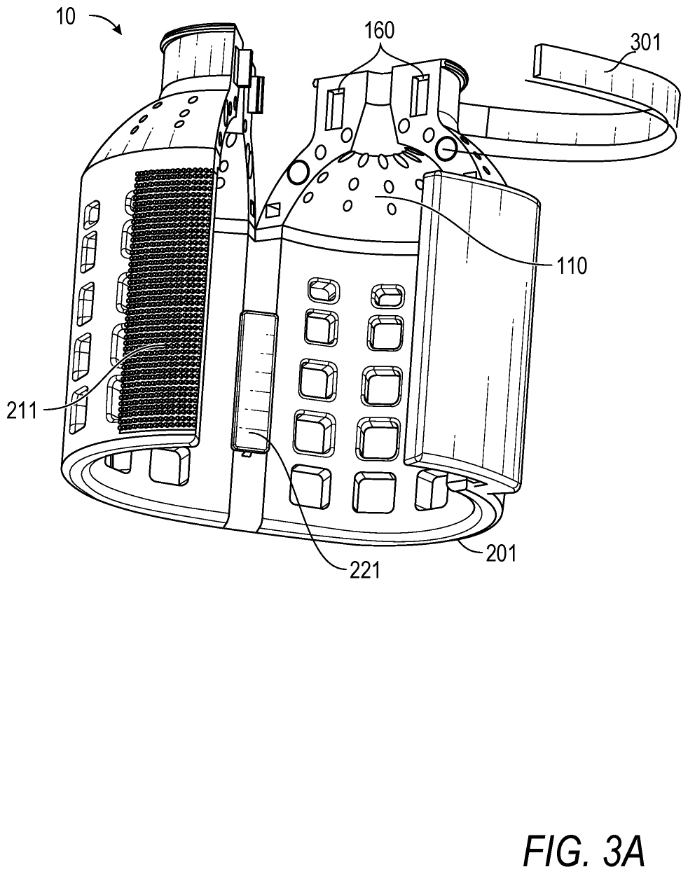

FIG. 3A is an illustration of a securement assembly 10 for a medical catheter in an open position, according to one embodiment of the present disclosure. The cap 100 of the securement assembly 10 can include one or more locking structures 160, wherein the one or more locking structures can be used to align and secure the two halves of the cap 100 together. In one embodiment, the locking structures 160 can include a male component and a female component, wherein the male component on a first half of the cap can fit into a female component on a second half. The male component can be, for example, a protrusion on the inner wall of a first half. The female component can be, for example, a slot or receptor in the inner wall of a second half. In one embodiment, the locking structures 160 can be a snap-fit joint, wherein a pushing force is applied to fit the male component into the opening of the female component. When the male component is fit into the female component, a pulling force must be applied to remove the male component from the opening of the female component. The pushing and the pulling forces slightly distort or deform the male component to fit through the opening of the female component. In one example, the opening of the female component can have approximately the same dimensions as the male component. The male component fits snugly inside the female component and is not separated from the female component until the pulling force is applied. The cap 100 can include one or more types of locking structures. The locking structures can be located along the inner walls of the cap, including the inner walls of the base 110.

In one embodiment, the anchor point of the body 200 can be a T-slot anchor 221. In one embodiment, the T-slot anchor 221 can be attached to an inner surface or layer of the body 200. The T-slot anchor 221 can be used to align and attach an inner layer to the body 200. In one embodiment, the T-slot anchor 221 can also be used to align the first half of the outer structure of the body 200 with the second half of the outer structure of the body 200. The first half and the second half can pivot around the axis of rotation formed by the T-slot anchor 221. The securement assembly 10 can transition from an open position, as pictured in FIG. 3A, to a closed position based on the position of the two halves. In one embodiment, the inner walls of the body 200 can include one or more locking mechanisms similar to locking structures 160 of the cap 100. The locking mechanisms of the body 200 can be used to align and secure the first half of the body and the second half of the body in a closed position. In one embodiment, the fastening mechanism of the body 200 can be a strip of hook and loop fabric 211, as illustrated in FIG. 3A. In one embodiment, the fastening mechanism can be attached to a panel, such as a plastic panel, on the body 200.

In one embodiment, the inner surface of the body 200 can be lined with an inner layer 201. The inner layer 201 can be a sponge layer. The inner layer 201 can be more flexible and/or softer than the material of the body 200. In one embodiment, the inner layer 201 can be a biocompatible material. The body 200 of the securement assembly can be in contact with human skin when the assembly is attached to an organ such as the penis. The inner layer 201 can provide a more comfortable and hygienic fit of the body 200 against the skin. The inner layer 201 can provide friction with the skin to restrict movement without causing irritation to the skin. In one embodiment, the inner layer 201 can be removed and replaced between uses of the securement assembly. In some examples, the body 200 can include additional layers of padding to ensure a secure fit around the penis. According to one embodiment, the cap 100 and/or the body 200 can include holes or perforations in the walls of the cap 100 and/or the body 200. The holes can provide for air flow through the securement assembly to prevent buildup of moisture or heat on the skin. In one embodiment, the holes can be used to secure any inner layers that may be attached to the cap 100 and/or the body 200.

FIG. 3B is a top view of a securement assembly 10 for a medical catheter in an open position, according to one embodiment of the present disclosure. The cap 100 can include the locking structures 160. The cap 100 can also include an opening 170 in the center of the cap 100, wherein the medical catheter can be secured in the opening 170 when the cap is in a closed position. The opening 170 can be an opening of a cylindrical channel running through the cap 100, wherein the medical catheter can be held in place in the cylindrical channel when the cap is in a closed position. In one embodiment, the size of the opening 170 and the cylindrical channel can be dependent on the medical catheter. In one embodiment, the size of the opening 170 and/or the channel can be modified. For example, layers of padding or can be placed along the inner walls of the opening 170 to ensure a secure fit of the medical catheter. In one embodiment, the cap 100 can include a pin 150. A first end of the fastener 301 can be attached to the pin 150 as an anchor. As an example, the first end of the fastener 301 can be looped around the pin 150 or can be attached to the pin 150 using an adhesive. The length of the fastener 301 can be wrapped around the cap 100 to secure the cap in a closed position. In one embodiment, the body of the securement assembly 100 can also include a fastening mechanism 211. The fastening mechanism 211 can include a first strip of hook-and-loop fabric on the outer surface of a first half of the body 200 and a second strip of hook-and-loop fabric on the inner surface of a second half of the body 200. The two strips of fabric can be secured to each other when they are in contact with each other and when a force is applied to the strips of fabric.

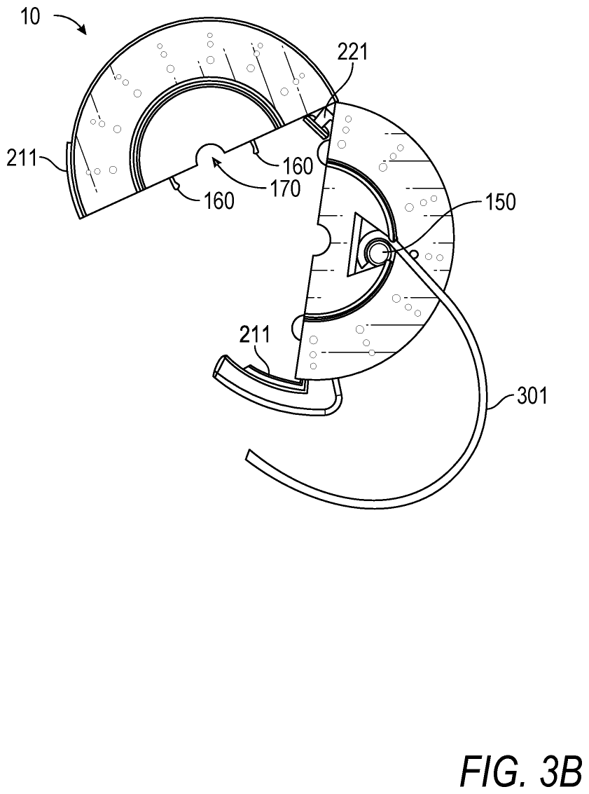

FIG. 4A is a cross section of one half of the cap 100 of the securement assembly for a medical catheter, according to one embodiment of the present disclosure. The cap 100 can include an approximately cylindrical tip 180 and a rounded base 110. The tip 180 of the cap can include the locking structures 160 and the opening 170 for the medical catheter. The base 110 of the cap 100 can have a rounded dome shape with a larger diameter than the diameter of the cylindrical tip 180. Alternative shapes and structures of the cap 100 are compatible with the securement assembly. For example, the cap 100 can be cylindrical or can be an angular structure. In one embodiment, the base 110 can include alignment features 190. The alignment features 190 can be used to align the first half of the cap with the second half of the cap. In one example, the alignment features 190 can include one or more cavities in the inner wall of a first half of the cap. The inner wall of the second half of the cap can include one or more protrusions matching the shape and dimensions of the one or more cavities in the first half. The alignment features 190 can guide a user to align the two halves properly so that the protrusions fit into the cavities. In one example, the cavities can be round and the protrusions can be spherical in shape, as illustrated in FIG. 4A.

In one embodiment, the inner surface of the cap 100 can be lined with a flexible layer such as a silicon layer 140. The silicon layer 140 can be more flexible and/or softer than the outer material of the cap. In one embodiment, the silicon layer 140 can be a biocompatible silicon. The base 110 of the cap can be in contact with human skin when the securement assembly is attached to an organ such as the penis. The silicon layer 140 can provide a more comfortable and hygienic fit of the cap 100 around the penis. In one embodiment, the thickness of the silicon layer 140 or the number of silicon layers can depend on the anatomy of the patient. The silicon layer 140 can also provide friction between the tip 180 of the cap and the medical catheter inserted into the opening 170. The friction between the silicon layer 140 and the medical catheter can reduce movement of the medical catheter, especially movement in and out of the opening 170 that would cause irritation or trauma to the tip of the penis. In one embodiment, the outer surface of the cap 100 can also be lined with a flexible and/or biocompatible layer. In one embodiment, attachments can be placed on or inside the cap for a tighter and more secure fit of the penis or the medical catheter. For example, the securement assembly can be used to secure a medical catheter with a smaller diameter than the diameter of the opening 170 in the cap. A ring, such as a hard plastic ring or a flexible silicon ring, can be inserted into the opening 170 to narrow the opening and/or the channel extending through the tip of the cap. The narrower opening restricts movement of a thinner medical catheter. In one embodiment, the attachment or the inner walls of the cap can include any number of raised structures to increase friction between the cap and the object being secured. In one embodiment, the inner walls of the cap can include gripping structures to increase friction between the inner walls of the cap and the medical catheter secured by the cap. The gripping structures can include, for example, nubs, teeth, ridges, bumps, or similar raised structures along the inner wall of the cap. In one embodiment, the gripping structures can be protrusions inside of the opening of the cap or the tip of the cap. The gripping structures can be in contact with the medical catheter when the medical catheter is inserted in the cap and can prevent or limit translational as well as rotational movement of the medical catheter.

FIG. 4B is an illustration of the cap 100 of the securement assembly for a medical catheter, according to one embodiment of the present disclosure. The cap 100 can include connectors to secure the cap to the body of the securement assembly. In one embodiment, the connectors can include holes 120 in the walls of the cap 100. In some embodiments, the holes 120 can be located along the base of the cap. Additionally or alternatively, the connectors can include hooks 130 along the bottom edge of the cap. As an exemplary embodiment, the hooks 130 can be inserted into slots at or near the top edge of the body of the securement assembly to connect the cap and the body. In one embodiment, the connectors can be used to attach and secure the silicon layer 140 of FIG. 4A to the inner surface of the cap 100. For example, the silicon layer 140 can include a ridged pattern, wherein the ridges of the silicon layer can fit into the holes 120 along the base of the cap. The fit of the ridges into the holes 120 can secure the silicon layer 140 against the inner wall of the cap 100. In one embodiment, the connectors can be used to secure the silicon of the body to the cap. The silicon of the body can include the outer material of the body, the internal wall of the body, or any additional layers of the body of the securement assembly.

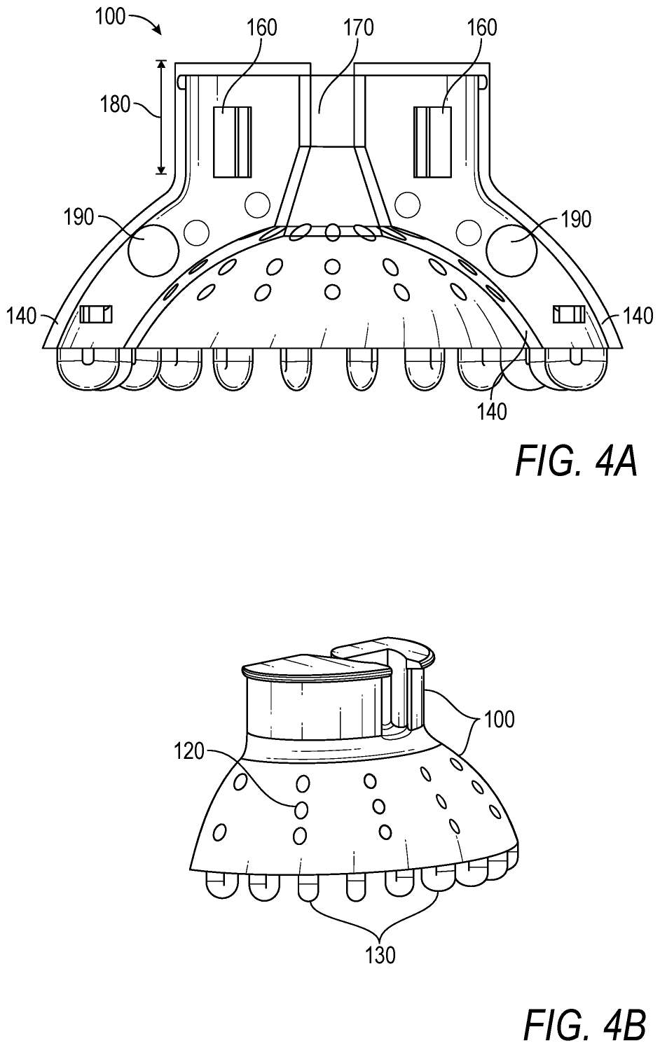

FIG. 5 is an illustration of an internal padding layer 231 for the body of the securement assembly for a medical catheter, according to one embodiment of the present disclosure. The body of the securement assembly can be a flexible material such as silicon to provide a comfortable fit and a range of motion, including bending or turning, for the patient when the securement assembly is attached to the patient. However, the fit of the securement assembly is dependent on patient anatomy. For example, if the securement assembly is too large, there can be movement of the organ within the securement assembly, causing irritation to the patient. If the securement assembly is too small, the tight fit will also cause irritation to the patient. Both scenarios can also result in unwanted movement of the medical catheter. There is therefore a need to adjust the internal diameter of the securement assembly to ensure a good fit for different patients. In one embodiment, an internal padding layer 231 can be attached to the body of the securement assembly. The padding layer 231 can be, for example, a sponge padding or a silicon padding. In one embodiment, the padding layer 231 can be biocompatible. In some embodiments, the padding layer 231 can include more than one layers of material. Padding layers 231 of varying thicknesses can be available for attachment to the body of the securement assembly. A padding layer 231 of appropriate thickness can be selected and attached to the securement assembly based on the anatomy of the patient. Advantageously, the padding layer 231 can be removed and replaced with a layer of a different thickness at a later time for a different patient.

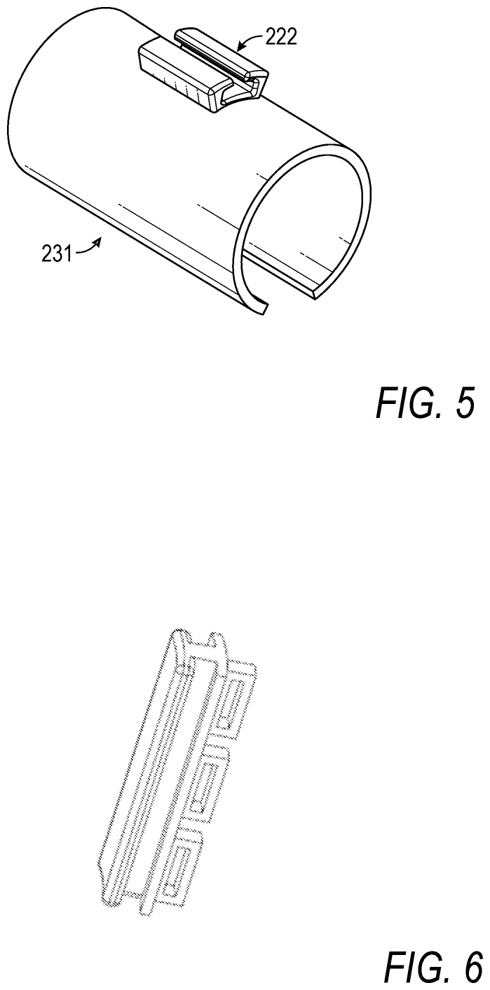

In one embodiment, the padding layer 231 can be attached to the body of the securement assembly using an anchor. The padding layer 231 can be attached to a track 222, wherein the track 222 can fit around an anchor attached to the body of the securement assembly. In one embodiment, the anchor can be the T-slot anchor 221 illustrated in FIG. 3A. The track 222 can be a corresponding T-slot track. The track 222 can slide over the anchor 221 to secure the padding layer 231 against the internal wall of the body of the securement assembly. FIG. 6 is an illustration of a T-slot anchor, according to one embodiment of the present disclosure. The T-slot anchor can be attached to the inner wall of the body of the securement assembly. In an exemplary embodiment, the anchor can include hooks, wherein the hooks can be inserted into receptor slots in the body of the securement assembly. In one embodiment, the anchor can be attached to the inner wall of the body of the securement assembly using an adhesive. In one embodiment, the body of the securement assembly can be formed with the anchor as a single component. According to one example, the bottom (base) of the anchor can be wider than the width of the track. The wider base of the anchor can hold the track in place when the track is fit over the anchor and prevent the track from sliding off of the anchor. Alternative or additional structural features can be included to ensure a secure fit of the track over the anchor and to prevent slipping or movement of the padding layer 231 after the track 222 is fitted over the anchor 221. The track and anchor enable the easy removal and replacement of the padding layer 231 if a different layer is needed for a patient.

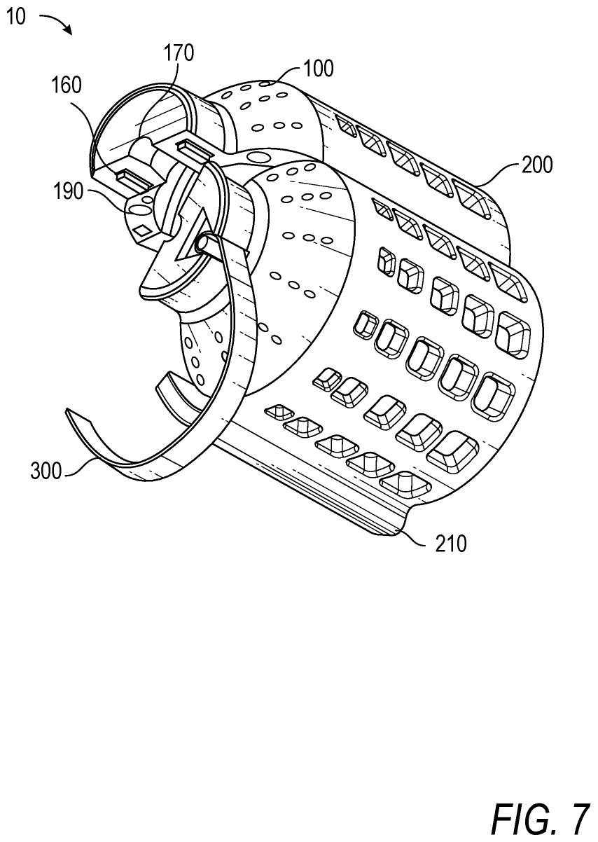

FIG. 7 is a side view of a securement assembly 10 for a medical catheter, according to one embodiment of the present disclosure. The securement assembly 10 is in an open position, wherein the two halves of the body 200 and the two halves of the cap 100 are attached at one end and separated at an opposing end. The tip of the cap 100 includes a locking structure 160 and an alignment structure 190. The locking structure and the alignment structure can be used to secure the cap in a closed position. When the cap is in a closed position, a medical catheter can be secured in the opening 170, the opening 170 leading to a channel running through the cap. The securement assembly can include a cap fastener 300 and a body fastener 210. The cap fastener 300 can wrap around the outside of the cap 100 when the cap is in a closed position to secure the cap. The body fastener 210 can attach a first half of the body to a second half of the body when the body is in a closed position to secure the body.

The securement assembly can be fabricated using a number of known manufacturing processes, including, but not limited to, 3D printing, injection molding, and silicon casting. In one embodiment, a model or mold of each half of the body of the securement assembly can be fabricated. For example, the model or mold can be printed. In one embodiment, the model or mold can encompass the cap of the securement assembly. The cap can be fabricated using any known plastics manufacturing process. The cap can be inserted into the model or mold, and the two halves can be secured together in a closed position to create a cavity for the silicon that forms the body of the securement assembly. The silicon can be poured into the model or mold and can form the body of the securement assembly and an inner layer of the cap. The silicon can be a curable silicon. The fastening mechanisms can be attached to the securement assembly after the cap and the body have been formed. In one embodiment, the connectors can be attached between the cap and the body. In one embodiment, the poured silicon can form the hooks 130 between the cap, which is inserted into the mold, and the body. The hooks 130 can fix the cap and the body together permanently when the assembly is formed. In one embodiment, the body and the cap can move as a single unit. For example, once the body and the cap are formed and assembled, the two halves of the body and the two halves of the cap open and close in tandem with each other.

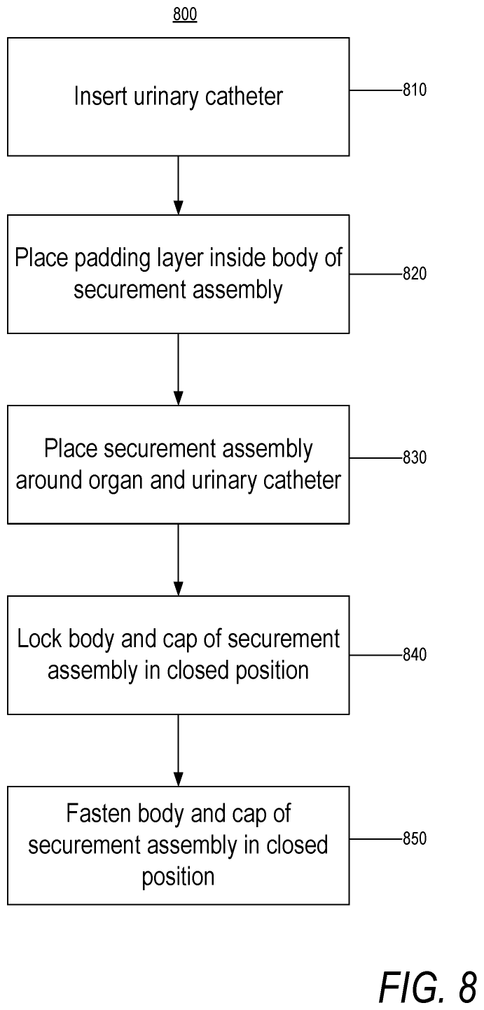

FIG. 8 is a method 800 for using a securement assembly for a urinary catheter inserted into a penis, according to one embodiment of the present disclosure. The urinary catheter can be inserted into the penis and threated into the bladder through the urethra in step 810. A layer of sponge padding can be selected and fitted into the inside of the body of the securement assembly in step 820 to ensure a proper fit around the penis. The securement assembly can be placed around the penis in step 830. The securement assembly can be positioned such that the urinary catheter fits into the opening in the tip of the cap of the securement assembly. The body and the cap of the securement assembly can be closed in step 840. A pushing force can be applied to the cap to lock the locking mechanisms. The fastening mechanism of the cap can be wrapped around the cap and fastened in step 850 to secure the cap in a closed position around the urinary catheter. The securement assembly can be left on the penis until the urinary catheter needs to be removed or replaced. In one embodiment, the cap of the securement assembly can be unfastened and opened to provide access to the urinary catheter at a later point.

The securement assembly of the present disclosure can be compatible with various medical catheters and insertion sites. For example, the securement assembly can secure straight medical catheters, bent or curved medical catheters, curled (pigtail) medical catheters, etc. The medical catheters can be inserted into an orifice in the body or through an incision in the skin on any part of the body. Advantageously, the securement assembly can be attached to the patient and to the medical catheter after the medical catheter has been inserted. The securement assembly does not interfere with the insertion of various types of medical catheters into the body. The body of the securement assembly can be configured to attach to any part of the skin for securement of a medical catheter.

In one embodiment, the securement assembly can be used to secure a medical catheter inserted into the chest. A thoracic catheter can be inserted into the skin and through the chest wall to drain fluid or air from the intrathoracic space surrounding the lung. The securement assembly can be used to secure thoracic catheters with varying sizes, fenestrations, shapes, suction types, and material compositions. For example, the securement assembly can secure right-angle thoracic catheters, which are common for draining the pleural cavity, or the space between the chest wall and the lung. A right-angle or approximately right-angle bend in the thoracic catheter can provide better access to areas of fluid buildup. The thoracic catheter can be connected to an evacuation system outside of the body. The evacuation system can apply a force to the thoracic catheter to encourage the fluid in the body to drain out of the body through the thoracic catheter in a unidirectional manner.

Placement and management of a thoracic catheter is important for preventing bleeding, injury to internal organs, or dislodgment of the thoracic catheter. A thoracic catheter must be fixed in place to avoid injury to nerves or vascular elements in the intercostal space, where the thoracic catheter is inserted. The depth of placement of a thoracic catheter is also important. Certain thoracic catheters include fenestrations along the length of the thoracic catheter near the drain opening to increase fluid drainage. The fenestrations should remain inside the body for proper drainage. Movement of the thoracic catheter can result in the drainage opening of the thoracic catheter moving away from the site of fluid buildup, resulting in an ineffective drainage system. In addition, thoracic catheters require regular dressing changes, which must be done without disturbing the placement of the thoracic catheter.

In some embodiments, the securement assembly of the present disclosure can be used to secure a thoracic catheter inserted into the chest. The cap of the securement assembly can hold the thoracic catheter while the body of the securement assembly can be attached to the chest. In one embodiment, the body of the securement assembly can be a flat base, wherein the base can adhere to the skin of the chest at the insertion site of the thoracic catheter. In one embodiment, the base can act as a dressing or covering for a wound (e.g., the insertion site) or the area surrounding a wound. The cap and the body can be removably coupled together to secure the thoracic catheter. In one embodiment, the body can be affixed to the chest while the cap can be removed to adjust the thoracic catheter or change a wound dressing. The cap of the securement assembly can be configured to hold a straight catheter or a bent catheter, including a right-angle catheter. Certain embodiments of the securement assembly can be configured to hold a low-profile catheter, which can also be referred to as a button.

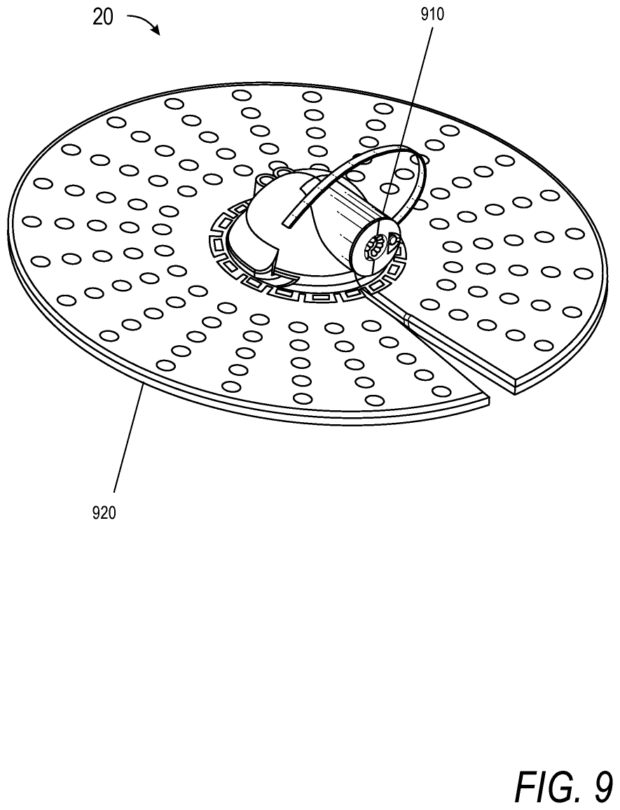

FIG. 9 is an illustration of a securement assembly 20 for a medical catheter, according to one embodiment of the present disclosure. The securement assembly 20 of FIG. 9A can be used to secure a right-angle thoracic catheter inserted into the chest. The cap 910 of the securement assembly can be a rigid plastic component. The cap 910 can include one or more fastening mechanisms to hold the cap 910 in a closed position around the medical catheter. The cap 910 can be removably coupled to the body 920 of the securement assembly. The body 920 can be an approximately circular pad. The body 920 can be affixed to a patient's chest. In one embodiment, the bottom surface of the body 920 can be a biocompatible adhesive material such that the body 920 can be adhered to human skin without the need for additional fixtures such as sutures. The cap 910 and the body 920 can include one or more locking mechanisms, aligners, guides, and the like to secure the cap 910 and the body 920 to each other.

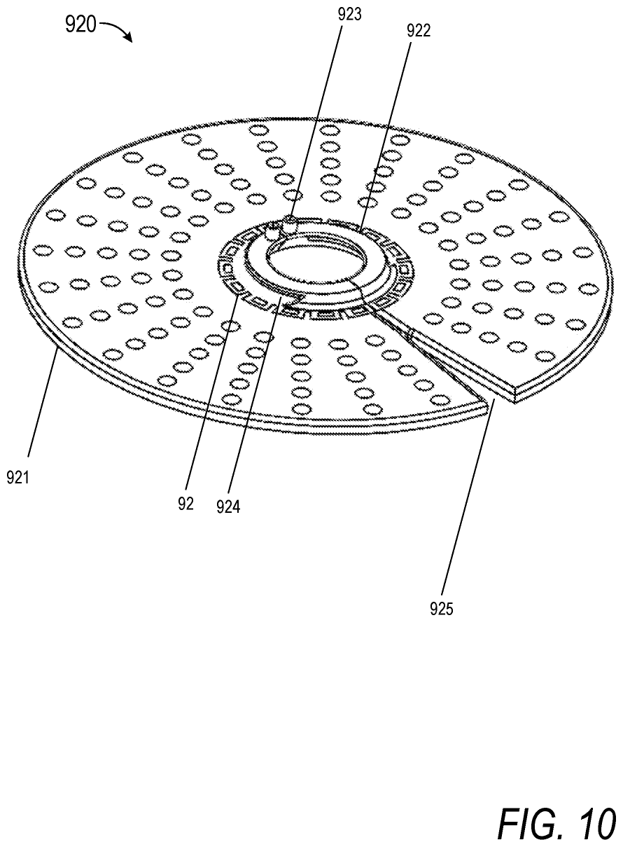

FIG. 10 is an illustration of a body 920 for a securement assembly 20 for a medical catheter, according to one embodiment of the present disclosure. The body 920 can be used for any medical catheter that is inserted through the skin, including, but not limited to, a thoracic catheter inserted into the chest. The size and shape of the body 920 can be dependent on where the medical catheter is inserted. In one embodiment, the body 920 can include a pad 921 that is affixed to the patient to secure the medical catheter. The bottom surface of the pad 921 or the bottom and top surface of the pad 921 can be adhesive. The pad 921 can include one or more pores to allow air flow to the skin underneath. The body 920 can include a hole in the center of the body 920 for insertion of the medical catheter and attachment of the cap. In one embodiment, the body 920 can include a dock 922, wherein the cap can be attached to the dock 922. The dock 922 can be positioned around the hole in the center of the body. In one embodiment, the dock 922 can be the same material as the cap. The dock 922 can be affixed to the pad 921 using an adhesive or an array of hooks or pins. For example, the base of the dock 922 can include an array of hooks that can be inserted into holes in the flexible pad 921 to secure the dock 922 in place. As an example, an array of holes 92 in the pad surrounding the dock 922 is illustrated in FIG. 10. Hooks, pins, or other structures in the base of the dock can be inserted into the array of holes 92 to fix the dock 922 to the pad 921. In one embodiment, the array of hooks or other insertable structures can be arranged on the pad and can be inserted into a corresponding array of holes in the base of the dock 922. In one embodiment, the dock 922 can be hot-pressed to the pad 921 or otherwise affixed to the pad by a pressure and/or temperature-activated mechanism.

The dock 922 can include one or more locking or guiding mechanisms for alignment and attachment of the cap. The shape and size of the dock can be approximately the shape and size of the bottom edge (base) of the cap. In one embodiment, the dock 922 can include a pin or a set of pins 923 for alignment with the cap. The pins can extend vertically upwards from the dock 922. The pins 923 can be inserted into corresponding slots in the cap when the cap is placed on the dock 922. The pins 923 can limit the translational movement of the cap once the cap is aligned on the dock 922. In one embodiment, the pins 923 can form axes of rotation for the halves of the cap. In one embodiment, the dock 922 can include one or more docking structures. The docking structure can be used to lock the cap and the dock together. The docking structure can include, for example, a sliding fit, a snap-fit joint, or a snap fastener. In one embodiment, the docking structure can include one or more slots 924 along the outer edge of the dock, as illustrated in FIG. 10. The slots 924 can run along a bottom portion of the outer edge of the dock 922 such that the top surface of the dock remains a continuous surface. In one embodiment, the dock 922 can be assembled from two halves. For example, the dock 922 can be assembled from two semi-circle halves that are fitted together to form the circular dock. The two halves can be secured together using an adhesive, or any of the fastening or locking mechanisms that have been described herein.

In one embodiment, the pad 921 can be composed of a medical-grade, biocompatible material, such as a biocompatible silicon. In one embodiment, the pad 921 can be a medical-grade, adhesive material. For example, the pad 921 can be a commercially available material used for wound dressing or other medical treatment related to the skin. The pad 921 can be used to cover a wound in the skin without causing irritation to the skin or the wound. In one embodiment, the bottom surface of the pad 921 can be a biocompatible, flexible adhesive, such as a biocompatible adhesive silicon. The bottom surface of the pad 921 can be a layer of a first material that is affixed to the body of the pad, the body of the pad being a second material. For example, the body of the pad can be a rigid plastic and the bottom surface of the pad can be a flexible silicon layer. In one embodiment, the pad 921 can include one or more pores to allow air flow to the skin underneath or may have no pores.

In one embodiment, the pad 921 can include a slit or cutout 925, as shown in FIG. 10. The cutout can extend through the dock 922. The body 920 of the securement assembly can be affixed to the patient after the medical catheter has already been inserted into the skin by fitting the cutout 925 in the pad around the medical catheter until the medical catheter is positioned in the hole in the center of the body 920. The cutout 925 provides access to the medical catheter and the hole in the center of the body 920 without having to thread the pad 921 over the entire length of the medical catheter after the medical catheter has been inserted. The body 920 can similarly be removed easily while leaving the medical catheter inserted in the skin by maneuvering the cutout 925 around the medical catheter. The pad can be easily removed and replaced frequently as a wound dressing or to allow access to the skin underneath the pad.

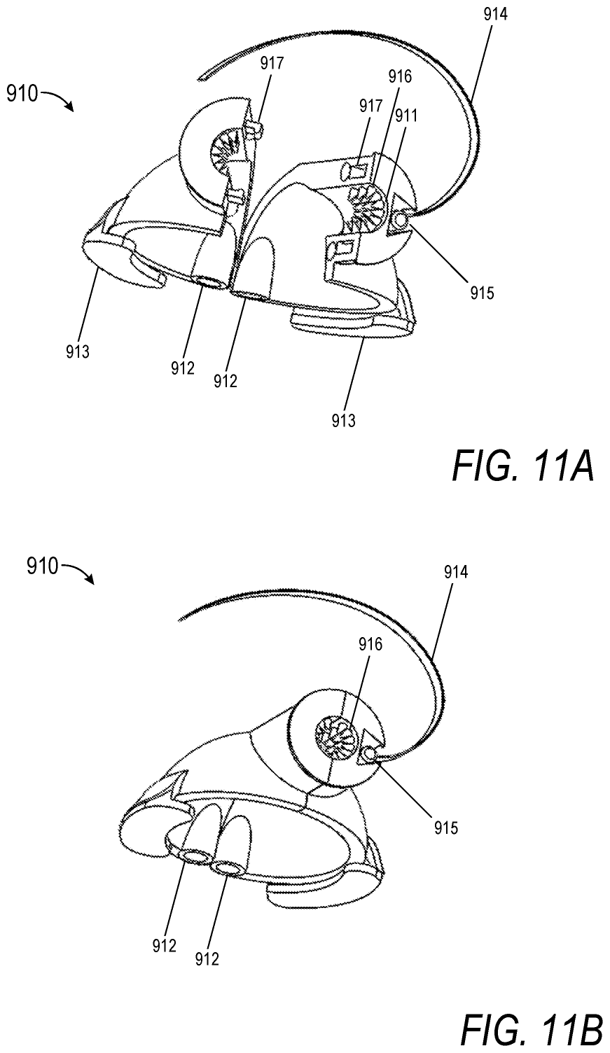

FIG. 11A is an illustration of a cap 910 for a securement assembly 20 for a medical catheter in an open position, according to one embodiment of the present disclosure. The cap 910 of FIG. 11A can be used to secure a medical catheter with a bend, such as a right angle thoracic catheter. In some embodiments, the cap 910 can be a domed cap. Alternative shapes and structures of the cap 910 are compatible with the securement assembly. For example, the cap 910 can be cylindrical or can be an angular structure. The opening 911 of the cap where the medical catheter is secured can be approximately perpendicular to the base of the cap. In some embodiments, the angle of the opening 911 can vary based on the medical catheter being secured. The cap 910 can be composed of two halves, wherein the two halves can open and close in order to secure the medical catheter. The two halves can be affixed together at a joint or can remain separate components. In one embodiment, each half can include an alignment structure 912 to align the cap on the dock of the securement assembly. The alignment structure can be, for example, a slot or a track that can be fitted over a structure on the body of the securement assembly. For example, the alignment structure 912 can be a cylindrical slot that can be fitted over the pin 923 on the dock 922 of the securement assembly illustrated in FIG. 10. The alignment structure 912 can limit the translational movement of the cap once the cap is aligned on the dock 922. In one embodiment, each half of the cap can rotate around the axis of the alignment structure 912 fitted onto the dock. The alignment structure 912 can form a hinge for the cap 910 to be opened and closed.

In one embodiment, each half of the cap 910 can include a docking structure 913 that can fit into a corresponding structure on the dock 922 to lock the cap 910 to the dock 922 in a closed position. In one embodiment, the docking structure 913 can be an overhanging piece that can be fitted into a slot in the dock. In one embodiment, the docking structure 913 can be a snap-fit mechanism. A pushing force can be applied to the cap to slightly distort or deflect the overhang 913 so that the overhang 913 fits into the slot 924 of the dock illustrated in FIG. 10. The pushing force can be applied to the side of the cap 910 in a lateral direction approximately parallel to the plane formed by the pad 921. The overhang 913 is secured in the slot 924 and will not move unless a similar force is applied in an opposite, outward (pulling) direction to slightly distort or deflect the overhang 913 and remove the overhang from the slot 924. In one embodiment, each half of the cap can include the docking structure 913. Each half can be locked to the dock independently.

In one embodiment, the cap 910 can be directly attached to the dock via the docking structure 913. The docking structure 913 on each half of the cap 910 can be aligned with the corresponding structure 924 on the dock and fitted into the dock to attach the cap to the dock. Each half of the cap can also be directly removed from the dock by removing the docking structure 913 from the corresponding structure 924 of the dock. In this manner, only a single step of translational motion is needed to attach and remove the cap rather than an alignment and fitting of the alignment structure followed by the locking of the cap to the dock. The translational motion can be approximately parallel to the pad 921 rather than a vertical motion needed to align the alignment structure of the cap to the pin of the dock. The single step can be advantageous for quicker attachment and removal of the cap.

In one embodiment, the cap 910 of the securement assembly 20 can include a fastener 914. In one embodiment, the fastener 914 can be a strip of fabric, flexible plastic, or similar material, wherein the strip can wrap around the top of the cap 910 to secure the first half of the cap and the second half of the cap together. In some embodiments, the fastener 914 can be an elastic strip. The fastener 914 can include a fastening mechanism such as hook-and-loop fabric, an adhesive, a button, a snap fastener, a pin or rivet, or the like in order to secure the cap 910 in a closed position wherein the first half and the second half of the cap are aligned and in contact. The fastener 914 can ensure that the medical catheter is secured tightly in the opening 911 in the center of the cap 910 when the cap is in the closed position. The opening 911 can be an opening of a cylindrical channel running through the cap, wherein the medical catheter can be held in place in the cylindrical channel when the cap is in a closed position. The tight fit of the cap around the medical catheter can reduce unwanted movement or bending of the medical catheter. In one embodiment, the cap 910 can include a pin 915. A first end of the fastener 914 can be attached to the pin 915 as an anchor. As an example, the first end of the fastener 914 can be looped around the pin 915 or can be attached to the pin 915 using an adhesive. The length of the fastener 914 can be wrapped around the cap 910 to secure the cap in a closed position.

In one embodiment, the inner walls of the cap can include gripping structures to increase friction between the inner walls of the cap and the medical catheter secured by the cap. The gripping structures can include, for example, nubs, teeth, ridges, bumps, or similar raised structures along the inner wall of the cap. In one embodiment, the gripping structures 916 can be protrusions inside of the opening 911 of the cap, as illustrated in FIG. 11A. The gripping structures can be in contact with the medical catheter when the medical catheter is inserted in the cap and can prevent or limit translational as well as rotational movement of the medical catheter.

In one embodiment, the cap 910 can include internal alignment features. The internal alignment features can be used to align the first half of the cap with the second half of the cap. In one example, the internal alignment features can include one or more cavities in the inner wall of a first half of the cap. The inner wall of the second half of the cap can include one or more protrusions matching the shape and dimensions of the one or more cavities in the first half. The internal alignment features can guide a user to align the two halves properly such that the protrusions fit into the cavities. In one example, the cavities can be round and the protrusions can be spherical in shape.

The cap 910 of the securement assembly 20 can include one or more locking structures 917, wherein the one or more locking structures can be used to align and secure the two halves of the cap 910 together. In one embodiment, the locking structures 917 can include a male component and a female component, wherein the male component on a first half of the cap can fit into a female component on a second half. The male component can be, for example, a protrusion on the inner wall of a first half. The female component can be, for example, a slot or receptor in the inner wall of a second half. In one embodiment, the locking structures 917 can include a snap-fit joint, wherein a pushing force is applied to fit the male component into the opening of the female component. When the male component is fit into the female component, a pulling force must be applied to remove the male component from the opening of the female component. The pushing and pulling forces slightly distort or deform the male component to fit through the opening of the female component. In one example, the opening of the female component can have approximately the same dimensions as the male component. The male component fits snugly inside the female component and is not separated from the female component until the pulling force is applied. The cap 910 can include one or more types of locking structures. The locking structures can be located along the inner walls of the cap, including near the opening in the cap for securing the medical catheter.

FIG. 11B is a bottom view of the cap 910 of a securement assembly 20 for a medical catheter in a closed position, according to an embodiment of the present invention. The locking structures along the inner wall of the cap 910 can be used to align and secure the two halves of the cap together in the closed position. For example, the locking structures can fit together such that there is no gap between the first half and the second half of the cap when the cap is in a closed position. The gripping structures 916 along the inside of the opening 911 of the cap can restrict movement of a medical catheter being secured by the cap 910. In one embodiment, the size of the opening and/or the size of the gripping structures can depend on the diameter and thickness of the medical catheter being secured. The fastener 914 can be attached to the cap at an anchor 915 along the outer wall of the cap. The fastener 914 can wrap around the opening of the cap at least once to hold the cap in the closed position. In one embodiment, the fastener 914 can include a self-adhering material or a self-affixing mechanism such as hook-and-loop fabric along the length of the fastener 914. The fastener 914 can be fastened without the need for additional structural elements on the cap. The alignment structures 912 can be adjacent to each other when the cap is in a closed position. The halves of the cap can swivel open and closed while the alignment structures 912 are attached to the dock.

FIG. 11C is a side view of the cap 910 of a securement assembly for a medical catheter in a closed position, according to one embodiment of the present invention. The docking structure 913 can be located at the edge of the cap. In one embodiment, each half of the cap can include a docking structure 913. Each half of the cap can also include an alignment structure 912, wherein the alignment structure 912 can fit over a corresponding structure on the dock. The alignment structure 912 can be, for example, a hollow cylinder. Each half of the cap can be attached and secured to the dock independently.

In one embodiment, the cap 910 of the securement assembly can form a cavity around the catheter and the insertion site. For example, there can be a cavity formed by the base of the cap between the cap and the skin. In one embodiment, the cavity can be filled with a wound dressing or an antibacterial agent. The cap can contain the dressing or other application within the cavity. In one embodiment, attachments can be placed on or inside the cap for a tighter and more secure fit around the medical catheter. For example, the securement assembly can be used to secure a medical catheter with a smaller diameter than the diameter of the opening in the cap. A ring, such as a hard plastic ring or a flexible silicon ring, can be inserted into the opening to narrow the opening and/or the channel extending through the tip of the cap. The narrower opening restricts movement of a thinner medical catheter. In one embodiment, the attachment can include any number of raised structures to increase friction between the attachment and the object being secured.

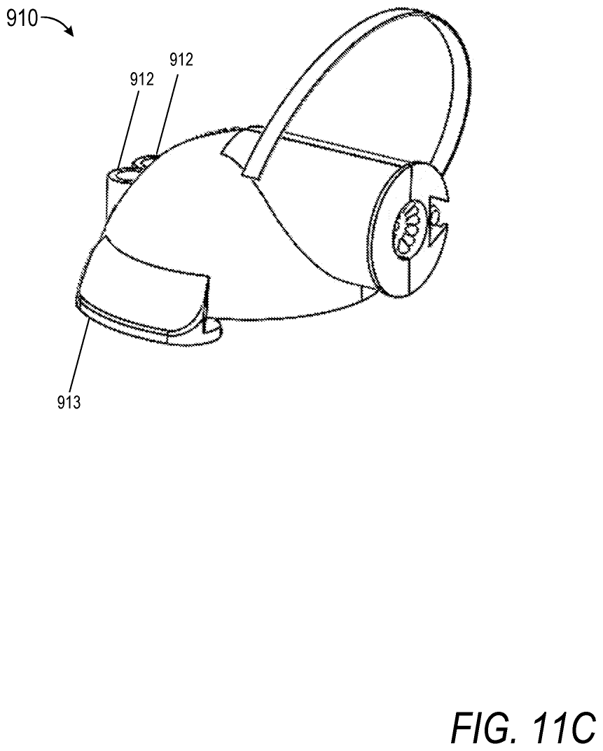

In some exemplary embodiments, the securement assembly of the present disclosure can be used to secure a catheter that is inserted into the abdomen, such as a suprapubic catheter or a drain. A suprapubic catheter is a type of urinary catheter that is inserted directly into the bladder through the skin rather than through the penis. Catheters can also be inserted into the abdomen to drain fluid in the abdominal cavity or the peritoneal cavity. FIG. 12 is an illustration of a securement assembly 30 for a medical catheter, according to one embodiment of the present disclosure. The securement assembly 30 can be used to secure a medical catheter that is approximately straight (rather than bent) at the insertion site and throughout the length that is external to the body. The securement assembly 30 can include a cap 1210 and a body 1220. The cap 1210 of the securement assembly can be a rigid plastic component. The cap 1210 can include one or more fasteners to hold the cap 1210 in a closed position around the medical catheter. The cap 1210 can be removably coupled to the body 1220 of the securement assembly. The body 1220 can be affixed to a patient's chest. In one embodiment, the body 1220 can be a biocompatible adhesive material such that the body 1220 can be adhered to human skin without the need for additional fixtures such as sutures. The body 1220 can be an approximately circular pad. The cap 1210 and the body 1220 can include one or more locking mechanisms, aligners, guides, and the like to secure the cap 1210 and the body 1220 to each other.

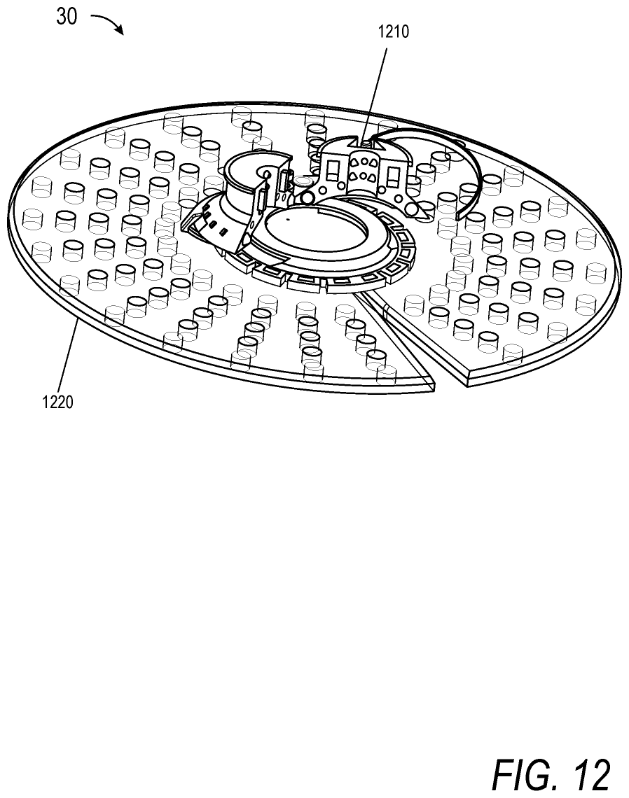

FIG. 13 is an illustration of a body 1220 for a securement assembly 30 for a medical catheter, according to one embodiment of the present disclosure. The body 1220 can be used for any medical catheter that is inserted through the skin, including, but not limited to, an abdominal catheter. The size and shape of the body 1220 can be dependent on where the medical catheter is inserted. In one embodiment, the body 1220 can include a pad 1221 that is affixed to the patient at or around the insertion site to secure the medical catheter. The bottom surface of the pad 1221 or the bottom and top surfaces of the pad 1221 can be adhesive. The pad 1221 can include one or more pores to allow air flow to the skin underneath or may have no pores. The body 1220 can include a hole in the center of the pad 1221 for insertion of the medical catheter and attachment of the cap. In one embodiment, the body 1220 can include a dock 1222, wherein the cap can be attached to the dock. The dock 1222 can be positioned around the hole in the center of the pad 1221. In one embodiment, the dock 1222 can be the same material as the cap, such as a rigid plastic. The dock 1222 can include one or more locking or guiding mechanisms for alignment and attachment of the cap. The shape and size of the dock can be approximately the shape and size of the bottom edge (base) of the cap.

In one embodiment, the dock 1222 can include a pin or a set of pins 1223 for alignment with the cap. The pins can extend vertically upwards from the dock 1222. The pins 1223 can be inserted into corresponding slots in the cap when the cap is placed on the dock 1222. The pins 1223 can limit the translational movement of the cap once the cap is aligned on the dock 1222. In one embodiment, the pins 1223 can form axes of rotation for the cap. In one embodiment, the dock 1222 can include one or more docking structures. The docking structure can be used to lock the cap and the dock together. The docking structure can include, for example, a sliding fit, a snap-fit joint, or a snap fastener. In one embodiment, the docking structure can include one or more slots 1224 along the outer edge of the dock, as illustrated in FIG. 13. The slots 1224 can run along a bottom portion of the outer edge of the dock 1222 such that the top surface of the dock is a continuous surface. In one embodiment, the dock 1222 can be assembled from two halves. For example, the dock 1222 can be assembled from two semi-circle halves that are fitted together to form the circular dock. The two halves can be secured together using an adhesive, or any of the fastening or locking mechanisms that have been described herein.

In one embodiment, the dock 1222 can be attached to a pad 1221 to form the body 1220 of the securement assembly. The dock 1222 can be affixed to the pad 1221 using an adhesive or a physical attachment, such as an array of hooks or pins. For example, the base of the dock 1222 can include an array of hooks that can be inserted into holes in the flexible pad 1221 to secure the dock 1222 in place. As an example, an array of holes 1226 in the pad surrounding the dock 1222 is illustrated in FIG. 13. Hooks, pins, or other structures in the base of the dock can be inserted into the array of holes 1226 to fix the dock 1222 to the pad 1221. In one embodiment, the array of hooks or other insertable structures can be arranged on the pad and can be inserted into a corresponding array of holes in the base of the dock 1222. In one embodiment, the dock 922 can be hot-pressed to the pad 921 or otherwise affixed to the pad by a pressure and/or temperature-activated mechanism.

In one embodiment, the pad 1221 can be composed of a medical-grade, biocompatible material, such as a biocompatible silicon. The pad 1221 can be flexible. In one embodiment, the pad 1221 can be a medical-grade, adhesive material. For example, the pad 1221 can be a commercially available material used for wound dressing or other medical treatment related to the skin. The pad 1221 can be used to cover a wound in the skin without causing irritation to the skin or the wound. In one embodiment, the bottom surface of the pad 1221 can be a biocompatible, flexible material, such as a biocompatible silicon. The bottom surface of the pad 1221 can be a layer of a first material that is affixed to the body of the pad, the body of the pad being a second material. For example, the body of the pad can be a rigid plastic and the bottom surface of the pad can be a flexible silicon layer. In one embodiment, the pad 1221 can include pores to allow for airflow to the skin through the pad 1221.

In one embodiment, the pad 1221 can include a slit or cutout 1225, as shown in FIG. 13. The cutout 1225 can extend through the dock 1222. The body 1220 of the securement assembly can be affixed to the patient after the medical catheter has already been inserted into the skin by fitting the cutout in the pad around the medical catheter until the medical catheter is positioned in the hole in the center of the body 1220. The cutout 1225 provides access to the medical catheter and the hole in the center of the body 1220 without having to thread the pad 1221 over the entire length of the medical catheter after the medical catheter has been inserted. The body 1220 can similarly be removed easily while leaving the medical catheter inserted in the skin by maneuvering the cutout around the medical catheter. The pad can be easily removed and replaced frequently as a wound dressing or to allow access to the skin underneath the pad.

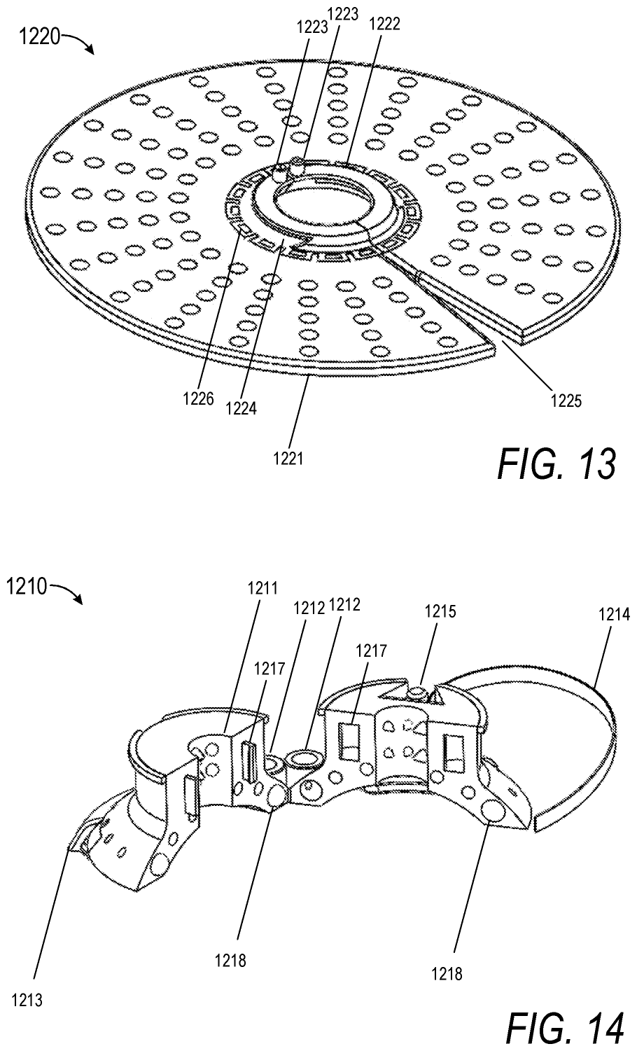

FIG. 14 is an illustration of a cap 1210 of a securement assembly for a medical catheter, according to one embodiment of the present disclosure. The cap 1210 can be used to secure a straight catheter. In one embodiment, the cap 1210 can be a domed cap. Alternative shapes and structures of the cap 1210 are compatible with the securement assembly. For example, the cap 1210 can be cylindrical or can be an angular structure. The opening 1211 of the cap 1210 where the medical catheter is secured can be coaxial with the base of the cap and the dock in the body of the securement assembly. The cap 1210 can be composed of two halves, wherein the two halves can open and close in order to secure the medical catheter. The two halves can be affixed together at a joint or can remain separate components. In one embodiment, each half can include an alignment structure 1212 to align the cap on the dock of the securement assembly. The alignment structure can be, for example, a slot or a track that can be fitted over a structure on the body of the securement assembly. For example, the alignment structure 1212 can be a cylindrical slot that can be fitted over the round pin 1223 on the dock 1222 of the securement assembly illustrated in FIG. 13. The alignment structure 1212 can limit the translational movement of the cap once the cap is aligned and attached to the dock 1222. In one embodiment, each half of the cap can rotate around the axis of the alignment structure 1212 fitted onto the dock. The alignment structure 1212 can form a hinge for the cap 1210 to be opened and closed.

In one embodiment, the cap 1210 can include a docking structure 1213 that can fit into a corresponding structure on the dock 1222 to lock the cap 1210 to the dock 1222 in a closed position. In one embodiment, the docking structure 1213 can be an overhanging piece that can be fitted into a corresponding slot in the dock. In one embodiment, the docking structure 1213 can be a snap-fit mechanism. A pushing force is applied to the cap to slightly distort or deflect the overhang 1213 so that the overhang 1213 fits into the slot 1224 of the dock illustrated in FIG. 13. The pushing force can be applied to the side of the cap 1210 in a lateral direction approximately parallel to the plane formed by the pad 1221. The overhang 1213 is secured in the slot 1224 and will not move unless a similar force is applied in an opposite, outward (pulling) direction to slightly distort or deflect the overhang 1213 and remove the overhang from the slot 1224. In one embodiment, each half of the cap can include the docking structure 1213. Each half can be locked to the dock independently.

In one embodiment, the cap 1210 can be directly attached to the dock via the docking structure 1213. The docking structure 1213 on each half of the cap 1210 can be aligned with the corresponding structure 1224 on the dock and fitted into the dock to attach the cap to the dock. Each half of the cap can also be directly removed from the dock by removing the docking structure 1213 from the corresponding structure 1224 of the dock. In this manner, only a single step of translational motion is needed to attach and remove the cap rather than an alignment and fitting of the alignment structure followed by the locking of the cap to the dock. The translational motion can be approximately parallel to the pad 1221 rather than a vertical motion needed to align the alignment structure of the cap to the pin of the dock. The single step can be advantageous for quicker attachment and removal of the cap.

In one embodiment, the cap 1210 of the securement assembly can include a fastener 1214. In one embodiment, the fastener 1214 can be a strip of fabric, flexible plastic, or similar material, wherein the strip can wrap around the top of the cap 1210 to secure the first half of the cap and the second half of the cap together. In some embodiments, the fastener 1214 can be an elastic strip. The fastener 1214 can include a fastening mechanism such as hook-and-loop fabric, an adhesive, a button, a snap fastener, a pin or rivet, or the like in order to secure the cap 1210 in a closed position wherein the first half and the second half of the cap are aligned and in contact. The fastener 1214 can ensure that the medical catheter is secured tightly in the opening 1211 in the center of the cap 1210 when the cap is in the closed position. The opening can be an opening of a cylindrical channel running through the cap, wherein the medical catheter can be held in place in the cylindrical channel when the cap is in a closed position The tight fit of the cap around the medical catheter can reduce unwanted movement or bending of the medical catheter. In one embodiment, the cap 1210 can include a pin 1215. A first end of the fastener 1214 can be attached to the pin 1215 as an anchor. As an example, the first end of the fastener 1214 can be looped around the pin 1215 or can be attached to the pin 1215 using an adhesive. The length of the fastener 1214 can be wrapped around the cap 1210 to secure the cap in a closed position.

In one embodiment, the inner walls of the cap can include gripping structures to increase friction between the inner walls of the cap and the medical catheter secured by the cap. The gripping structures can include, for example, nubs, teeth, ridges, bumps, or similar raised structures along the inner wall of the cap. In one embodiment, the gripping structures can be protrusions and can be located inside of the opening 1211 of the cap. The gripping structures can be in contact with the medical catheter when the medical catheter is inserted in the cap and can prevent or limit translational as well as rotational movement of the medical catheter.

In one embodiment, the cap 1210 can include internal alignment features 1218. The internal alignment features can be used to align the first half of the cap with the second half of the cap. In one example, the internal alignment features can include one or more cavities in the inner wall of a first half of the cap. The inner wall of the second half of the cap can include one or more protrusions matching the shape and dimensions of the one or more cavities in the first half. The internal alignment features can guide a user to align the two halves properly such that the protrusions fit into the cavities. In one example, the cavities can be round and the protrusions can be spherical in shape, as in the internal alignment features 1218 of FIG. 14.

The cap 1210 of the securement assembly 30 can include one or more locking structures 1217, wherein the one or more locking structures can be used to align and secure the two halves of the cap 1210 together. In one embodiment, the locking structures 1217 can include a male component and a female component, wherein the male component on a first half of the cap can fit into a female component on a second half. The male component can be, for example, a protrusion on the inner wall of a first half. The female component can be, for example, a slot or receptor in the inner wall of a second half. In one embodiment, the locking structure 1217 can be a snap-fit joint, wherein a pushing force is applied to fit the male component into the opening of the female component. When the male component is fit into the female component, a pulling force must be applied to remove the male component from the opening of the female component. The pushing and pulling forces slightly distort or deform the male component to fit through the opening of the female component. In one example, the opening of the female component can have approximately the same dimensions as the male component. The male component fits snugly inside the female component and is not separated from the female component until the pulling force is applied. The cap 1210 can include one or more types of locking structures. The locking structures can be located along the inner walls of the cap, including near the opening in the cap for securing the medical catheter.

In one embodiment, the cap 1210 of the securement assembly can form a cavity around the catheter and the insertion site. For example, there can be a cavity formed by the base of the cap between the cap and the skin. In one embodiment, the cavity can be filled with a wound dressing or an antibacterial agent. The cap can contain the dressing or other application within the cavity. In one embodiment, attachments can be placed on or inside the cap for a tighter and more secure fit around the medical catheter. For example, the securement assembly can be used to secure a medical catheter with a smaller diameter than the diameter of the opening in the cap. A ring, such as a hard plastic ring or a flexible silicon ring, can be inserted into the opening to narrow the opening and/or the channel extending through the tip of the cap. The narrower opening restricts movement of a thinner medical catheter. In one embodiment, the attachment can include any number of raised structures to increase friction between the attachment and the object being secured.

The shape and dimensions of the cap can be configured to secure various medical catheters. According to some embodiments, the cap and/or the opening in the cap can be configured to fit around external structures attached to or integrated into a medical catheter. In one embodiment, the cap of the securement assembly can secure a low-profile medical catheter, such as a gastrostomy button. Low-profile medical catheters can be connected to structures inside the body, such as a balloon that can be used for feeding. In one embodiment, the cap can form a chamber in the shape of a low-profile medical catheter. For example, the cap can be shaped to fit around a safety plug, a port, and any other external features at the end of the low-profile medical catheter that emerges from the insertion site. In one embodiment, the cap can enclose the end of a low-profile medical catheter. The cap can fit around the low-profile medical catheter to prevent any movement of the external portions of the low-profile medical catheter. According to one implementation, the inner walls of the cap can be in contact with the external features of the low-profile medical catheter to reduce empty space surrounding the low-profile medical catheter. In one embodiment, a port of the medical catheter can be accessed while the medical catheter is secured in the cap of the securement assembly. Advantageously, the two halves of the cap can be separated to allow access to medical catheter and then rejoined to secure and protect the medical catheter.

The cap of a securement assembly for a medical catheter can be made using any plastic manufacturing method, including, but not limited to, 3D printing, injection molding, casting. In one embodiment, the dock can be made using a similar plastic manufacturing method. In one embodiment, the inner wall of a cap can be coated with a biocompatible layer, such as biocompatible silicon, in case there is contact between the cap and human skin. In one embodiment, the body of the securement assembly can be a flexible material, as has been described herein. In one embodiment, the body of the securement assembly can be a rigid material, such as polylactic acid (PLA) or acrylonitrile butadiene styrene (ABS), with a layer of flexible, biocompatible material forming the bottom surface of the body.

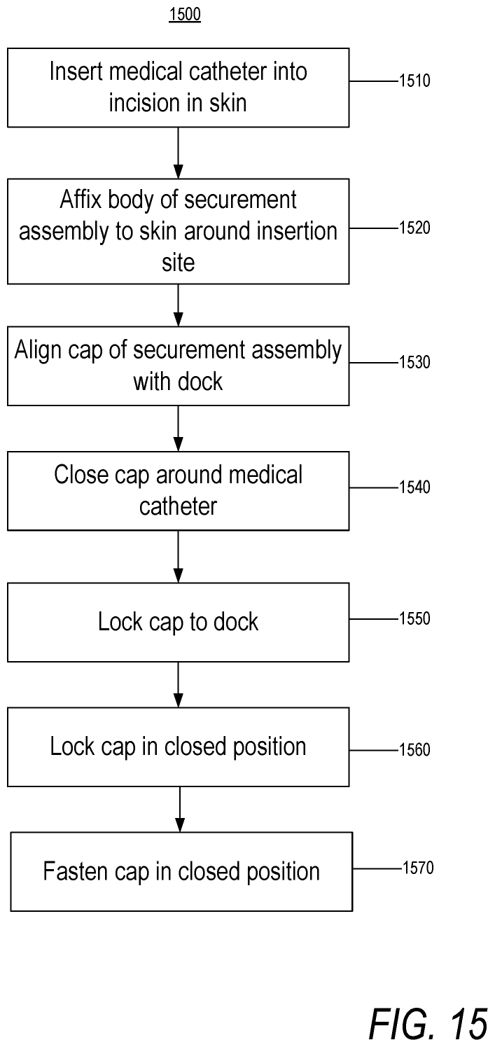

FIG. 15 is a method for using the securement assembly, according to one embodiment of the present disclosure. For example, the method 1500 of FIG. 15 can be used to secure a medical catheter that is inserted through an incision into the skin. The medical catheter can first be inserted into the skin in step 1510. The body of the securement assembly can be affixed to the patient's skin such that the medical catheter and the insertion site are located in the hole in the center of the pad. The body of the securement assembly can be affixed to the patient's skin without moving the medical catheter or touching the insertion site by fitting the cutout in the pad around the medical catheter until the medical catheter is in the center of the pad. The bottom surface of the pad can be a medical-grade adhesive material. Each half of the cap of the securement assembly can be attached to the dock in step 1520 by positioning the alignment structure of the cap over the alignment pin of the dock. The cap can be an open position when first attached to the dock. The halves of the cap can be closed around the medical catheter in step 1530 so that the medical catheter fits into the opening in the cap. The halves of the cap can be aligned by fitting the alignment structures in the inner wall of the cap together. The halves of the cap can be locked to the dock in a closed position in step 1540 by applying a pushing force to a docking structure, such as a snap-fit structure formed by the dock and the base of the cap. The halves of the cap can further be locked to each other in a closed position in step 1550 by applying a pushing force to a locking mechanism in the cap, such as a snap-fit structure formed by the two halves of the cap. The medical catheter is secured inside the locked cap. The fastening mechanism of the cap can be fastened in step 1560. For example, a strip of hook-and-loop fabric can be wrapped around the cap near the opening of the cap to fasten the opening of the cap. The securement assembly can be disassembled and removed at a later point.

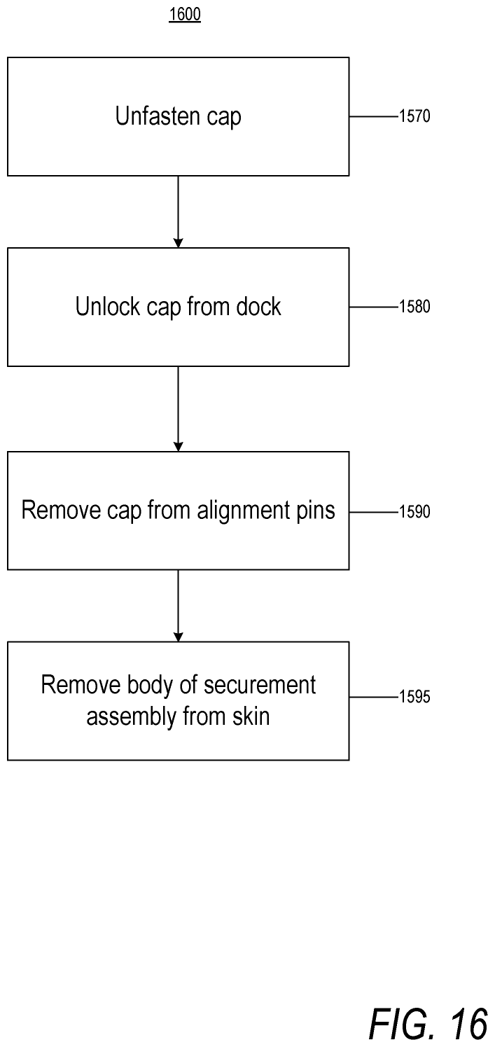

FIG. 16 is a method 1600 for removing the securement assembly. The securement assembly can be fully or partially removed in order to remove the medical catheter or treat the insertion site. The fastening mechanism of the cap can be unfastened in step 1570. The cap can be unlocked and opened by pulling the halves of the cap apart. The pulling force can separate the snap-fit structures formed by the two halves of the cap and the snap-fit structures formed by the base of the cap and the dock in step 1580. The cap can be left attached to the alignment pins of the dock and can rotate around the axes formed by the alignment pins to allow access to the medical catheter. In one embodiment, the cap can be removed from the dock by lifting the cap off of the alignment pins in step 1590. The medical catheter can be removed or replaced while the body of the securement assembly (the pad) is still affixed to the patient's skin. The body of the securement assembly can be removed from the patient if necessary by pulling the pad off of the patient's skin in step 1595. For example, the pad can be a wound dressing that is changed periodically.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals.

(1) A securement assembly for a medical catheter inserted into an organ, comprising a cylindrical body; and a domed cap removably coupled to the cylindrical body, the domed cap having a first half and an opposing second half, the first half and the opposing second half being removably coupled together to form a channel inside the domed cap terminating at an opening in the domed cap, the channel being configured to secure the medical catheter, and the cylindrical body forming a chamber inside of the cylindrical body, the chamber being configured to secure the organ.

(2) The securement assembly of (1), wherein the domed cap includes an internal locking structure, wherein the internal locking structure is configured to secure the first half of the domed cap against the opposing second half of the domed cap.

(3) The securement assembly of any (1) to (2), wherein the internal locking structure is a snap-fit joint.

(4) The securement assembly of any (1) to (3), wherein the domed cap includes a fastener configured to wrap around the first half of the domed cap and the opposing second half of the domed cap.

(5) The securement assembly of any (1) to (4), further comprising a layer of padding removably attached to an inner wall of the cylindrical body.

(6) The securement assembly of any (1) to (5), wherein the cylindrical body includes a first half and an opposing second half, wherein the first half of the cylindrical body is removably coupled to the opposing second half of the cylindrical body to form the chamber.

(7) The securement assembly of any (1) to (6), wherein an inner surface of the domed cap is lined with a silicon layer.

(8) A securement assembly for a medical catheter inserted into an organ, comprising a cylindrical body; and a domed cap removably coupled to the cylindrical body, the domed cap having a first half and an opposing second half, the first half and the opposing second half being removably coupled together to form a channel inside the domed cap terminating at an opening in the domed cap, the channel being configured to secure the medical catheter, the cylindrical body forming a chamber inside of the cylindrical body, the chamber being configured to secure the organ, and the domed cap being a first material and the cylindrical body being a second material, wherein the second material is more flexible than the first material.

(9) The securement assembly of (8), wherein the domed cap includes an internal locking structure, wherein the internal locking structure is configured to secure the first half of the domed cap against the opposing second half of the domed cap.

(10) The securement assembly of any (8) to (9), wherein the internal locking structure is a snap-fit joint.

(11) The securement assembly of any (8) to (10), wherein the domed cap includes a fastener configured to wrap around the first half of the domed cap and the opposing second half of the domed cap.

(12) The securement assembly of any (8) to (11), further comprising a layer of padding removably attached to an inner wall of the cylindrical body.

(13) The securement assembly of any (8) to (12), wherein the cylindrical body includes a first half and an opposing second half, wherein the first half of the cylindrical body is removably coupled to the opposing second half of the cylindrical body to form the chamber.

(14) The securement assembly of any (8) to (13), wherein the second material is a biocompatible material.

(15) A securement assembly for a medical catheter inserted into an organ, comprising a cylindrical body; and a domed cap removably coupled to the cylindrical body, the domed cap having a first half and an opposing second half, the first half and the opposing second half being removably coupled together to form a channel inside the domed cap terminating at an opening in the domed cap, the channel being configured to secure the medical catheter, the cylindrical body having a layer of padding forming an inner wall of the cylindrical body, and the cylindrical body forming a chamber inside of the cylindrical body, the chamber being configured to secure the organ.

(16) The securement assembly of (15), wherein the domed cap includes an internal locking structure, wherein the internal locking structure is configured to secure the first half of the domed cap against the opposing second half of the domed cap.

(17) The securement assembly of any (15) to (16), wherein the internal locking structure is a snap-fit joint.

(18) The securement assembly of any (15) to (17), wherein the cylindrical body includes a first half and an opposing second half, wherein the first half of the cylindrical body is removably coupled to the opposing second half of the cylindrical body to form the chamber.

(19) The securement assembly of any (15) to (18), wherein the first half of the cylindrical body and the opposing second half of the cylindrical body are removably attached to the layer of padding to form the chamber.

(20) The securement assembly of any (15) to (19) wherein the layer of padding is a biocompatible material.

(21) A securement assembly for a medical catheter inserted into an opening in skin, comprising a dock being attached to a base; and a domed cap removably coupled to the dock, the dock surrounding a cutout in the base, the domed cap having a first half and an opposing second half, the first half and the opposing second half being removably coupled together to form a channel inside the domed cap terminating at an opening in the domed cap, the channel being configured to secure the medical catheter, and the domed cap including a docking structure, wherein the docking structure fits into a slot in the dock to couple the domed cap to the dock.

(22) The securement assembly of (21), wherein the base is a biocompatible, adhesive pad.

(23) The securement assembly of any (21) to (22), wherein the docking structure includes an alignment pin, wherein the alignment pin fits into a cavity in the domed cap.

(24) The securement assembly of any (21) to (23), wherein the domed cap includes an internal locking structure, wherein the internal locking structure is configured to secure the first half of the domed cap against the opposing second half of the domed cap.

(25) The securement assembly of any (21) to (24), wherein the internal locking structure is a snap-fit joint.

(26) The securement assembly of any (21) to (25), wherein the channel inside the domed cap includes one or more protrusions extending from an inner wall of the domed cap into the channel.

(27) The securement assembly of any (21) to (26), wherein the base includes a second cutout, the second cutout extending from the dock to an edge of the base.

(28) A securement assembly for a medical catheter inserted into an opening in skin, comprising a dock being attached to a base; and a domed cap removably coupled to the dock, the dock surrounding a cutout in the base, the domed cap having a first half and an opposing second half, the first half and the opposing second half being coupled together to form a channel inside the domed cap terminating at an opening in the domed cap, the channel being configured to secure the medical catheter, the opening in the domed cap being perpendicular to the base, the domed cap including a docking structure, wherein the docking structure fits into a slot in the dock to couple the domed cap to the dock.

(29) The securement assembly of (28), wherein the base is a biocompatible, adhesive pad.

(30) The securement assembly of any (28) to (29), wherein the docking structure includes an alignment pin, wherein the alignment pin fits into a cavity in the domed cap.

(31) The securement assembly of any (28 to (30), wherein the domed cap includes an internal locking structure, wherein the internal locking structure is configured to secure the first half of the domed cap against the opposing second half of the domed cap.

(32) The securement assembly of any (28) to (31), wherein the internal locking structure is a snap-fit joint.

(33) The securement assembly of any (28) to (32), wherein the channel inside the domed cap includes one or more protrusions extending from an inner wall of the domed cap into the channel.

(34) The securement assembly of any (28) to (33), wherein the base includes a second cutout, the second cutout extending from the dock to an edge of the base.

(35) A securement assembly for a medical catheter inserted into an opening in skin, comprising a dock being attached to a base; and a domed cap removably coupled to the dock, the dock surrounding a cutout in the base, and the domed cap having a first half and an opposing second half, the first half and the opposing second half being removably coupled together to form a channel inside the domed cap terminating at an opening in the domed cap, the channel being configured to secure the medical catheter, the domed cap being a first material and the base being a second material, wherein the second material is more flexible than the first material.

(36) The securement assembly of (35), wherein the base is a biocompatible, adhesive pad.

(37) The securement assembly of any (35) to (36), wherein the dock is the first material.

(38) The securement assembly of any (35) to (37), wherein the domed cap includes an internal locking structure, wherein the internal locking structure is configured to secure the first half of the domed cap against the opposing second half of the domed cap.

(39) The securement assembly of any (35) to (38), wherein the channel inside the domed cap includes one or more protrusions extending from an inner wall of the domed cap into the channel.

(40) The securement assembly of any (35) to (39), wherein the base includes a second cutout, the second cutout extending from the dock to an edge of the base.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A securement assembly for a medical catheter inserted into an organ, comprising:
a cylindrical body; and
a domed cap removably coupled to the cylindrical body,
the domed cap having a first half and an opposing second half, the first half and the opposing second half being removably coupled together to form a channel inside the domed cap terminating at an opening in the domed cap, the channel being configured to secure the medical catheter, and
the cylindrical body forming a chamber inside of the cylindrical body, the chamber being configured to secure the organ.

2. The securement assembly of claim 1, wherein the domed cap includes an internal locking structure, wherein the internal locking structure is configured to secure the first half of the domed cap against the opposing second half of the domed cap.

3. The securement assembly of claim 2, wherein the internal locking structure is a snap-fit joint.

4. The securement assembly of claim 1, wherein the domed cap includes a fastener configured to wrap around the first half of the domed cap and the opposing second half of the domed cap.

5. The securement assembly of claim 1, further comprising a layer of padding removably attached to an inner wall of the cylindrical body.

6. The securement assembly of claim 1, wherein the cylindrical body includes a first half and an opposing second half, wherein the first half of the cylindrical body is removably coupled to the opposing second half of the cylindrical body to form the chamber.

7. The securement assembly of claim 1, wherein an inner surface of the domed cap is lined with a silicon layer.

8. A securement assembly for a medical catheter inserted into an organ, comprising:
a cylindrical body; and
a domed cap removably coupled to the cylindrical body,
the domed cap having a first half and an opposing second half, the first half and the opposing second half being removably coupled together to form a channel inside the domed cap terminating at an opening in the domed cap, the channel being configured to secure the medical catheter,
the cylindrical body forming a chamber inside of the cylindrical body, the chamber being configured to secure the organ, and
the domed cap being a first material and the cylindrical body being a second material, wherein the second material is more flexible than the first material.

9. The securement assembly of claim 8, wherein the domed cap includes an internal locking structure, wherein the internal locking structure is configured to secure the first half of the domed cap against the opposing second half of the domed cap.

10. The securement assembly of claim 9, wherein the internal locking structure is a snap-fit joint.

11. The securement assembly of claim 8, wherein the domed cap includes a fastener configured to wrap around the first half of the domed cap and the opposing second half of the domed cap.

12. The securement assembly of claim 8, further comprising a layer of padding removably attached to an inner wall of the cylindrical body.

13. The securement assembly of claim 8, wherein the cylindrical body includes a first half and an opposing second half, wherein the first half of the cylindrical body is removably coupled to the opposing second half of the cylindrical body to form the chamber.

14. The securement assembly of claim 8, wherein the second material is a biocompatible material.

15. A securement assembly for a medical catheter inserted into an organ, comprising:
a cylindrical body; and
a domed cap removably coupled to the cylindrical body,
the domed cap having a first half and an opposing second half, the first half and the opposing second half being removably coupled together to form a channel inside the domed cap terminating at an opening in the domed cap, the channel being configured to secure the medical catheter,
the cylindrical body having a layer of padding forming an inner wall of the cylindrical body, and
the cylindrical body forming a chamber inside of the cylindrical body, the chamber being configured to secure the organ.

16. The securement assembly of claim 15, wherein the domed cap includes an internal locking structure, wherein the internal locking structure is configured to secure the first half of the domed cap against the opposing second half of the domed cap.

17. The securement assembly of claim 16, wherein the internal locking structure is a snap-fit joint.

18. The securement assembly of claim 15, wherein the cylindrical body includes a first half and an opposing second half, wherein the first half of the cylindrical body is removably coupled to the opposing second half of the cylindrical body to form the chamber.

19. The securement assembly of claim 18, wherein the first half of the cylindrical body and the opposing second half of the cylindrical body are removably attached to the layer of padding to form the chamber.

20. The securement assembly of claim 15, wherein the layer of padding is a biocompatible material.

21. A securement assembly for a medical catheter inserted into an opening in skin, comprising:
a dock being attached to a base; and
a domed cap removably coupled to the dock,
the dock surrounding a cutout in the base,
the domed cap having a first half and an opposing second half, the first half and the opposing second half being removably coupled together to form a channel inside the domed cap terminating at an opening in the domed cap, the channel being configured to secure the medical catheter, and
the domed cap including a docking structure, wherein the docking structure fits into a slot in the dock to couple the domed cap to the dock.

22. The securement assembly of claim 21, wherein the base is a biocompatible, adhesive pad.

23. The securement assembly of claim 21, wherein the docking structure includes an alignment pin, wherein the alignment pin fits into a cavity in the domed cap.

24. The securement assembly of claim 21, wherein the domed cap includes an internal locking structure, wherein the internal locking structure is configured to secure the first half of the domed cap against the opposing second half of the domed cap.

25. The securement assembly of claim 24, wherein the internal locking structure is a snap-fit joint.

26. The securement assembly of claim 21, wherein the channel inside the domed cap includes one or more protrusions extending from an inner wall of the domed cap into the channel.

27. The securement assembly of claim 21, wherein the base includes a second cutout, the second cutout extending from the dock to an edge of the base.

28. A securement assembly for a medical catheter inserted into an opening in skin, comprising:
a dock being attached to a base; and
a domed cap removably coupled to the dock,
the dock surrounding a cutout in the base,
the domed cap having a first half and an opposing second half, the first half and the opposing second half being coupled together to form a channel inside the domed cap terminating at an opening in the domed cap, the channel being configured to secure the medical catheter,
the opening in the domed cap being perpendicular to the base,
the domed cap including a docking structure, wherein the docking structure fits into a slot in the dock to couple the domed cap to the dock.

29. The securement assembly of claim 28, wherein the base is a biocompatible, adhesive pad.

30. The securement assembly of claim 28, wherein the docking structure includes an alignment pin, wherein the alignment pin fits into a cavity in the domed cap.

31. The securement assembly of claim 28, wherein the domed cap includes an internal locking structure, wherein the internal locking structure is configured to secure the first half of the domed cap against the opposing second half of the domed cap.

32. The securement assembly of claim 31, wherein the internal locking structure is a snap-fit joint.

33. The securement assembly of claim 28, wherein the channel inside the domed cap includes one or more protrusions extending from an inner wall of the domed cap into the channel.

34. The securement assembly of claim 28, wherein the base includes a second cutout, the second cutout extending from the dock to an edge of the base.

35. A securement assembly for a medical catheter inserted into an opening in skin, comprising:
a dock being attached to a base; and
a domed cap removably coupled to the dock,
the dock surrounding a cutout in the base, and
the domed cap having a first half and an opposing second half, the first half and the opposing second half being removably coupled together to form a channel inside the domed cap terminating at an opening in the domed cap, the channel being configured to secure the medical catheter,
the domed cap being a first material and the base being a second material, wherein the second material is more flexible than the first material.

36. The securement assembly of claim 35, wherein the base is a biocompatible, adhesive pad.

37. The securement assembly of claim 35, wherein the dock is the first material.

38. The securement assembly of claim 35, wherein the domed cap includes an internal locking structure, wherein the internal locking structure is configured to secure the first half of the domed cap against the opposing second half of the domed cap.

39. The securement assembly of claim 35, wherein the channel inside the domed cap includes one or more protrusions extending from an inner wall of the domed cap into the channel.

40. The securement assembly of claim 35, wherein the base includes a second cutout, the second cutout extending from the dock to an edge of the base.
